(19) United States
(12) Patent Application Publication
(10) Pub. No.: US 2023/0207055 A1
(43) Pub. Date: Jun. 29, 2023

(54) IDENTIFYING GENES WITH DIFFERENTIAL SELECTIVE CONSTRAINT BETWEEN HUMANS AND NON-HUMAN PRIMATES

(71) Applicant: Illumina, Inc., San Diego, CA (US)

(72) Inventors: Hong GAO, Palo Alto, CA (US); Joshua Goodwin Jon MCMASTER-SCHRAIBER, Berkeley, CA (US); Kai-How FARH, San Mateo, CA (US)

(21) Appl. No.: 18/147,214

(22) Filed: Dec. 28, 2022

Related U.S. Application Data
(60) Provisional application No. 63/294,820, filed on Dec. 29, 2021.

(51) Int. Cl. G16B 25/10; G16B 20/50

(52) U.S. Cl. CPC G16B 25/10; G16B 20/50

(57) ABSTRACT

The technology disclosed relates to identifying differential selective constraint on a gene-by-gene basis between a target species and one or more non-target species. The disclosed systems and methods can use a population genetics model wherein an average selection coefficient per gene per species is estimated and further applied to estimate selective constraint. The disclosed systems and methods can use a generalized linear mixed model wherein depletion of missense variants per gene per species is estimated and further applied to estimate selective constraint. In some cases, the disclosed systems and methods can use various combinations of the components from the population genetics model or the generalized linear mixed model to identify the intersection of genes classified as having differential selective constraint by numerous approaches for validation purposes.

### PRIORITY APPLICATIONS

This application claims the benefit of and priority to the following:

U.S. Provisional Patent Application No. 63/294,813, titled “PERIODIC MASK PATTERN FOR REVELATION LANGUAGE MODELS,” filed Dec. 29, 2021 (Attorney Docket No. ILLM 1063-1/IP-2296-PRV);

U.S. Provisional Patent Application No. 63/294,816, titled “CLASSIFYING MILLIONS OF VARIANTS OF UNCERTAIN SIGNIFICANCE USING PRIMATE SEQUENCING AND DEEP LEARNING,” filed Dec. 29, 2021 (Attorney Docket No. ILLM 1064-1/IP-2297-PRV);

U.S. Provisional Patent Application No. 63/294,820, titled “IDENTIFYING GENES WITH DIFFERENTIAL SELECTIVE CONSTRAINT BETWEEN HUMANS AND NON-HUMAN PRIMATES,” filed Dec. 29, 2021 (Attorney Docket No. ILLM 1065-1/IP-2298-PRV);

U.S. Provisional Patent Application No. 63/294,827, titled “DEEP LEARNING NETWORK FOR EVOLUTIONARY CONSERVATION,” filed Dec. 29, 2021 (Attorney Docket No. ILLM 1066-1/IP-2299-PRV);

U.S. Provisional Patent Application No. 63/294,828, titled “INTER-MODEL PREDICTION SCORE RECALIBRATION,” filed Dec. 29, 2021 (Attorney Docket No. ILLM 1067-1/IP-2301-PRV); and

U.S. Provisional Patent Application No. 63/294,830, titled “SPECIES-DIFFERENTIABLE EVOLUTIONARY PROFILES,” filed Dec. 29, 2021 (Attorney Docket No. ILLM 1068-1/IP-2302-PRV).

U.S. Provisional Patent Application No. 63/294,710, titled “APPROPRIATE REFERENCE GENOMES FOR TARGET SPECIES,” filed Dec. 29, 2021 (Attorney Docket No. IP-2300-PRV).

The priority applications are incorporated by reference as if fully set forth herein.

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using deep convolutional neural networks to analyze ordered data.

### INCORPORATIONS

The following are incorporated by reference for all purposes as if fully set forth herein:

Sundaram, L. et al. Predicting the clinical impact of human mutation with deep neural networks. Nat. Genet. 50, 1161-1170 (2018);

Jaganathan, K. et al. Predicting splicing from primary sequence with deep learning. Cell 176, 535-548 (2019);

U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);

U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);

U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);

U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);

U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);

U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);

U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US);

U.S. patent application Ser. No. 16/160,978, titled “DEEP LEARNING-BASED SPLICE SITE CLASSIFICATION,” filed on Oct. 15, 2018 (Attorney Docket No. ILLM 1001-4/IP-1680-US);

U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US);

U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US);

U.S. Patent Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-1/IP-2142-PRV);

U.S. Patent Application No. 63/175,767, titled “EFFICIENT VOXELIZATION FOR DEEP LEARNING,” filed on Apr. 16, 2021, (Atty. Docket No. ILLM 1048-1/IP-2143-PRV);

U.S. patent application Ser. No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US);

U.S. patent application Ser. No. 17/975,536, titled “MASK PATTERN FOR PROTEIN LANGUAGE MODELS,” filed on Oct. 27, 2022, (Atty. Docket No. IP-2296-US1);

U.S. patent application Ser. No. 17/947,049, titled “DEEP LEARNING NETWORK FOR EVOLUTIONARY CONSERVATION,” filed on Sep. 16, 2022, (Atty. Docket No. IP-2299-US);

U.S. Provisional Patent Application No. 63/253,122, titled “PROTEIN STRUCTURE-BASED PROTEIN LANGUAGE MODELS,” filed Oct. 6, 2021 (Attorney Docket No. ILLM 1050-1/IP-2164-PRV);

U.S. Provisional Patent Application No. 63/281,579, titled “PREDICTING VARIANT PATHOGENICITY FROM EVOLUTIONARY CONSERVATION USING THREE-DIMENSIONAL (3D) PROTEIN STRUCTURE VOXELS,” filed Nov. 19, 2021 (Attorney Docket No. ILLM 1060-1/IP-2270-PRV); and

U.S. Provisional Patent Application No. 63/281,592, titled “COMBINED AND TRANSFER LEARNING OF A VARIANT PATHOGENICITY PREDICTOR USING GAPED AND NON-GAPED PROTEIN SAMPLES,” filed Nov. 19, 2021 (Attorney Docket No. ILLM 1061-1/IP-2271-PRV).

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Interpreting the effects of human genetic variants and their impact on disease risk is a foundational component of personalized genomic medicine. Out of more than 70 million possible nonsynonymous variants in the human genome, less than one percent are annotated and the remainder are variants of unknown clinical significance. In particular, approaches for the classification of human genetic variants with rare frequency are of consequence, due to the correlation between variant rarity and variant pathogenicity. However, the analysis of rare variants is intrinsically limited by low frequency in the human population. One strategy for interpreting the clinical significance of human genetic variants involves the use of information from closely related primate species to infer the pathogenicity of orthologous human variants. By conducting population sequencing studies in closely related non-human primate species, some models can catalog common variants and rule these out as pathogenic in human, analogous to how sequencing more diverse human populations has helped to advance clinical variant interpretation.

The overlap of evolutionary biology and genetic medicine includes the detection of species-specific and lineage-specific evolutionary adaptation and selective pressures. As whole genome sequencing has increased in efficiency, biotechnology firms and researchers have sequenced whole genomes (or large percentages of genomes) for thousands of humans and several samples from primate and other vertebrate species. This data can be processed to uncover evidence for the origin of modern human genetic traits and the variation of these traits. In comparison to human populations, for instance, primates exhibit relatively fewer missense mutations-consistent with the majority of newly-arising human missense mutations being removed by natural selection due to their deleteriousness. Despite a common evolutionary lineage, humans and primates (or various other species) comprise genes that are subject to varying degrees of selective constraint. Consequently, the comparative study of evolutionary differences, such as comparative selective constraint, has a countless number of practical applications to augment the characterization of human genetic variants.

Because humans and certain primate species exhibit closely genetics and protein sequences, some pathogenicity models have been used to analyze related human and primate protein sequences to infer the pathogenicity of orthologous human variants. For example, end-to-end deep learning approaches for variant effect predictions are applied to predict the pathogenicity of missense variants from protein sequence and sequence conservation data (See Sundaram, L. et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. PrimateAI in particular uses sequences of wild-type and mutant proteins to compare the difference and decide the pathogenicity of mutations using the trained deep neural networks. Such an approach that utilizes the protein sequences for pathogenicity prediction is promising because it can avoid the problem of circularity and overfitting to previous knowledge. However, compared to the adequate number of data to train the deep neural networks effectively, the number of clinical data available in ClinVar is relatively small. To overcome this data scarcity, PrimateAI uses common human variants and variants from primates as benign data while simulated variants based on trinucleotide context were used as unlabeled data.

PrimateAI outperforms prior methods when trained directly upon sequence alignments. PrimateAI learns important protein domains, conserved amino acid positions, and sequence dependencies directly from the training data consisting of about 120,000 human samples. PrimateAI substantially exceeds the performance of other variant pathogenicity prediction tools in differentiating benign and pathogenic de-novo mutations in candidate developmental disorder genes, and in reproducing prior knowledge in ClinVar. These results suggest that PrimateAI is an important step forward for variant classification tools that may lessen the reliance of clinical reporting on prior knowledge.

Despite the utility of PrimateAI in predicting variant pathogenicity, PrimateAI's pathogenicity predictions do not directly exhibit or indicate different selective constraints exerted on genes of human and non-human primate (or other) species. Therefore, an opportunity arises to compare natural selection acting on individual genes across the primate lineage and identify genes with differential selective constraint in humans and non-human primates. More accurate variant pathogenicity prediction may result or be used to better evaluate pathogenicity predictions.

### BRIEF DESCRIPTION OF THE DRAWINGS

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which:

FIG. 1 is a schematic diagram of a system for detecting differential constraint via the comparison of selection coefficients, according to one embodiment of the disclosed technology.

FIG. 2 shows an example genetic sequence and corresponding variants resulting from a single nucleotide polymorphism.

FIG. 3A shows an example genetic sequence and corresponding variant effects resulting from a single nucleotide polymorphism.

FIG. 3B contrasts conserved positions with segregating sites within a group of allelic variants.

FIG. 4A shows a plurality of sample scenarios illustrating the relationship between natural selection and genetic variation, wherein the examples exhibit evidence for greater selective constraint.

FIG. 4B shows a plurality of sample scenarios illustrating the relationship between natural selection and genetic variation, wherein the examples exhibit evidence for relaxed selective constraint.

FIG. 5 shows the relationship between relative fitness and natural selection for an example gene.

FIG. 6A shows a phylogenic tree for a target species and non-target species orthologous to the target species.

FIG. 6B shows representative selection coefficients per gene per species.

FIG. 6C shows representative sample calculations of selective constraint per gene using selection coefficients as measures for selection.

FIG. 7A shows a set of mathematical functions configured to estimate an average population-scaled mutation rate across species.

FIG. 7B shows a set of mathematical functions configured to estimate an average population-scaled selection coefficient across species.

FIG. 7C shows a flow diagram comprising steps to correct an average population-scaled selection coefficient across species for demographic differences between species.

FIG. 8 shows a flow diagram comprising steps to identify genes where differential selective constraint is present between a target species and one or more non-target species using a likelihood-ratio test.

FIG. 9 shows a first example computer system that can be used to implement the technology disclosed, according to one embodiment of the disclosed technology.

FIG. 10 is a schematic diagram of a system for detecting differential constraint via the comparison of missense-to-synonymous ratios, according to one embodiment of the disclosed technology.

FIG. 11 shows representative sample calculations of selective constraint per gene using missense-to-synonymous ratios as measures for selection.

FIG. 12 shows a set of mathematical functions configured to estimate the relationship between missense depletion per gene in a target species and a plurality of non-target species.

FIG. 13 shows a flow diagram comprising steps to identify genes where differential selective constraint is present between a target species and one or more non-target species using a Z-test.

FIG. 14 shows a second example computer system that can be used to implement the technology disclosed, according to one embodiment of the disclosed technology.

FIG. 15 shows a plurality of performance metrics of a population genetic model fitting to human and primate data.

FIG. 16 shows the correlation of missense-to-synonymous ratios and selection coefficient estimates in humans and primates with pLI and s_het.

FIG. 17 shows the relationship of human and primate missense-to-synonymous ratio based on Poisson generalized linear mixed modeling.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

The technologies disclosed can be used to identify genes with differential selective constraint between orthologous species. The technology disclosed can be used to augment the study of clinical disease in a target species by comparing the selective forces acting on individual genes in the target species and other closely-related non-target species. Identifying genes with differential selective constraint between species is complicated by species-specific differences in diversity, demographic factors, allelic frequency, and sample size between species cohorts. To address these challenges, the methods and systems disclosed comprise a population genetic-based approach to estimate statistical measures of selective constraint and leverage the estimated statistical measures of selective constraint to determine if purifying selection against nonsynonymous mutations in a target species is stronger or weaker than observed in closely-related non-target species. Furthermore, the methods and systems disclosed comprise strategies to control for outlier genes that have an abnormal distribution of variants because of data quality issues.

As mentioned above, in some embodiments, a PrimateAI model (e.g., PrimateAI 1D, PrimateAI 2D, or PrimateAI 3D) or another variant pathogenicity prediction model comprises neural networks trained on variants of known pathogenicity for learning important protein domains, conserved amino acid positions, and sequence dependencies directly from the training data. In some cases, the training data comprises about 120,000 human samples. To substantiate or validate predictions from PrimateAI or another variant pathogenicity prediction model, the disclosed methods and systems can compare selective constraint across species. For instance, the disclosed systems can compare selective constraint for a target species (e.g., humans) against selective constraint for one or more non-target species (e.g., primates) to detect a difference between the species-specific measures of selective constraint (e.g., differential selective constraint). When the disclosed systems can detect genes with high measures of selective constraint (e.g., constrained genes) in both target (e.g., human) and non-target (e.g., primate) species, in some embodiments, the disclosed systems can validate the pathogenicity predictions of PrimateAI. In some embodiments, the disclosed methods and systems perform or utilize the techniques described in U.S. patent application Ser. No. 17/975,536 and/or U.S. patent application Ser. No. 17/947,049, both of which are incorporated by reference above.

To validate a pathogenicity prediction or a pathogenicity score for a variant, in some embodiments, the disclosed systems determine an indicator of a differential selective constraint for a gene of interest between a target species and one or more non-target species. Additionally, or alternatively, the disclosed systems determine a deviation metric to approximate a magnitude and a direction of differential selective constraint between a target species and one or more non-target species. By determining an indicator of differential selective constraint or a deviation metric, the disclosed systems provide a supplementary metric indicating a confidence or reliability of a pathogenicity prediction or pathogenicity score for a variant amino-acid sequence output by a PrimateAI model or another variant pathogenicity prediction model.

### INTRODUCTION

New applications of evolutionary biology and population genetics inform the characterization of genetic mechanisms contributing to human health outcomes. New, rapidly developing methods augment traditional genetic medicine with new contextual data on the evolutionary history of human traits to shed light on the phylogeny (e.g., evolutionary origin and comparative adaptation of traits throughout speciation) and adaptive significance (e.g., mechanism and interaction dynamics that aid in explaining why natural selection has not further depleted pathogenic alleles). The ability of these interdisciplinary studies to produce useful insights on phenomena that have long confounded the scientific community has established evolutionary medicine as a field of study in its own right.

While the increase in available biological sequence data has advanced the accuracy and efficiency of clinical variant interpretation, there are still tens of millions of possible protein-variants in the human genome with unknown clinical significance, many of which are rare variants with the potential to be highly penetrant and deleterious to health. Despite the high penetrance of rare deleterious variants, their rarity limits their ability to explain phenotypic variance to a small fraction of the population, since the majority of individuals do not carry a rare deleterious variant for a given phenotype. Further complicating matters, genetic disorders caused by variants in multiple genes (polygenic genetic disorders) outnumber those caused by variants in a single gene (monogenic genetic disorders). Hence, a polygenic genetic disorder characterized by a particular disease phenotype can often arise through a range of many non-overlapping genotype combinations. Similarly, a countless number of genetic disorders are observed within the human population with severely limited insight as to their genetic and molecular mechanism of pathogenesis. Characterization of some genetic disorders is limited by extensive polygenic complexity, such as major depressive disorder. Other genetic disorders are challenging to characterize due to their rarity in the population, such as ectodermal dysplasia. By contrast, in some cases, outlier genes can be an indication of a monogenic genetic disorder. By determining an indicator of differential selective constraint or a deviation metric, the disclosed systems provide an indicator or metric that facilitates determining recessive inheritance. For instance, the disclosed indicator of differential selective constraint or deviation metric can indicate when a gene of interest is not (or less) subject to selective constraint and, therefore, more likely to exhibit recessive alleles that can be inherited.

Evolutionary perspectives on genetic disease may provide scalable approaches for interpreting the effects of human genetic variants and their impact on disease risk by using information from closely-related primate species to infer the pathogenicity of orthologous human variants. In one potential application of the technology disclosed, human genetic variants can be mapped to a reference genome from a closely-related primate species. The pathogenicity of these human genetic variants can be inferred based on their allele frequencies in other primate populations. This technique can be applied to systematically catalog common variants and rule these out as unlikely to be pathogenic in humans, analogous to how sequencing more diverse human populations has advanced clinical variant interpretation.

However, pathogenicity within a closely-related primate species does not always correlate with pathogenicity in humans due to a plethora of differences between species, including both factors that are quite easy to observe (e.g., differences in environment, demography, and non-overlapping traits inherent to speciation) and more complex genetic factors (e.g., rates of background mutation or surrounding sequence context for a particular allele) that require more complex analysis. Thus, the extent to which evolutionarily-related species can be used to infer knowledge about a proximate species is limited by the extent to which one understands the evolutionary relationship between the cohort of species under analysis. Understanding how forces of natural selection exerted upon species influence the similarity and divergence of orthologous traits between species is a key component for understanding the evolutionary context for these traits. Hence, the comparison of selective constraint on a particular gene between orthologous species provides a rich source of information that is applicable to understanding the clinical significance of that particular gene.

In one or more embodiments, the methods and systems disclosed herein provide improvement or advantages over prior systems. For example, the disclosed methods and systems are more computationally efficient than prior systems. In some cases, to identify differential selection between a target species and a non-target species, some prior systems determine or calculate mutation rates for every gene of a target species and/or a non-target species. For genetically complex species, such as humans and other primates, calculating mutation rates for such large numbers of genes can be computationally expensive, requiring substantial processing power, memory, and computing time. By contrast, the methods and systems disclosed herein use an empirical phase approach to share information across multiple genes and/or species. Indeed, as described below, the disclosed methods and systems can implement a population genetic model that includes two phases: 1) modeling counts of synonymous segregating sites to learn a neutral background distribution of mutation rates per gene per species, and 2) applying the learned neutral background distribution to estimate the average selection per gene across species. By using the empirical phase approach, the disclosed methods and systems are more computationally efficient than prior systems, expending less processing power, memory, and computing time.

As set forth below, in certain implementations, the methods and systems disclosed comprise a technology for identifying differential selection between a target species and closely-related non-target species at gene resolution. In certain cases, the methods and systems disclosed further comprise a model configured to quantify the broad-scale similarity of natural selection between a target species and one or more closely-related non-target species and to identify genes evolving subject to significantly different selective pressure in the target species as compared to the cohort of closely-related non-target species. Moreover, the methods and systems disclosed may be configured to aggregate a plurality of non-target species into a cohort that can be described by a shared single statistical metric or a distribution of statistical metrics in some implementations. In other implementations, the disclosed model can be applied to a plurality of genes at species resolution to share information about mutation rate and data quality across genes. By sharing information about mutation rate and data quality across genes at species resolution, as well as sharing information about selective constraint and data quality across species at gene resolution, in some embodiments, the disclosed methods and systems can control for outlier genes that have an abnormal distribution of variants.

Consequently, the disclosed methods and systems can improve data quality over prior systems that make assumptions on quality of data fed into predictive models, such as PrimateAI. Indeed, because different species have different distributions of mutation rates, and because the disclosed systems and methods account for multiple species, the techniques employed by the disclosed methods and systems address data quality issues for data input into a model, resulting in more accurate predictions from models such as PrimateAI. In some embodiments, the disclosed methods and systems perform or utilize the techniques described in U.S. patent application Ser. No. 17/975,536 and/or U.S. patent application Ser. No. 17/947,049, both of which are incorporated by reference above. Thus, the methods and systems disclosed are applicable to a range of species populations of varying sample size, cohort size, and data quality. Indeed, in some embodiments, the disclosed methods and systems generate and/or apply a different, unique model for each different target species, such as humans, chimpanzees, orangutans, or some other primate (or non-primate) target species. For instance, if the disclosed methods and systems are applies to generate selection coefficients (or to determine selective constraint) for sixty different target species, the methods and systems would generate and apply sixty different population genetic models.

In one implementation of the technology disclosed, differential selective constraint per gene between the target species and non-target species is determined by estimating the population-scaled selection coefficient per gene in the target species and the average population-scaled selection coefficient per gene across a cohort of non-target species, followed by applying a likelihood ratio test to determine if selection against protein-changing mutations is different between the target species and the non-target species. In another implementation of the technology disclosed, differential selective constraint per gene between the target species and non-target species is determined by estimating the relationship between the depletion of protein-changing mutations in the target species and the depletion of protein-changing mutations in the cohort of non-target species, followed by analysis to determine if the depletion of protein-changing mutations in the target species is significantly different from what is expected based on the depletion of protein-changing mutations in the cohort of non-target species. In many implementations, a combination of both described approaches for estimating selective constraint are used for more robust validation.

The following disclosure is organized as follows. First, a brief summary of the terminology used herein is given as a guide to aid in navigating the description of the technology disclosed. Next, a first system that can be used to implement the technology disclosed is introduced, according to one embodiment of the technology disclosed. To further elaborate upon the input and output data types described, a plurality of exemplary genetic sequences and related sample calculations are presented, followed by a description of one approach by which the disclosed technology employs these genetic concepts and methodologies to detect differential selective constraint between species, in accordance with the first system presented.

A second system is then introduced that can also be used to implement the technology disclosed, according to another embodiment of the technology disclosed. Again, a plurality of exemplary genetic sequences and related sample calculations are presented as a briefing prior to describing another approach by which the disclosed technology is used to detect differential selective constraint between species, in accordance with the second system presented.

Finally, a representative sample of performance results obtained by various embodiments of the technology disclosed are detailed as objective indica of inventiveness and non-obviousness.

### Terminology

The description herein refers to a plurality of implementations of the disclosed methods and systems, wherein a target species and a cohort of non-target species are compared to identify differential selective constraint between orthologous genes. Definitions of terminology, terminology to be understood as synonymous, and terminology not to be understood as synonymous will now be presented.

A target species is a species of interest under analysis by the technology disclosed. A variety of objectives may influence the selection of a species as the target species, such as the relevance of the selected species to a particular field of study within medicine, ecology, and so on. In many implementations, the target species is selected to be human due to the significant clinical implication of human genetic variants. However, the target species need not always be human and neither the selected species nor the reasoning for selection of a target species should be considered a limitation of the technology disclosed. Correspondingly, a non-target species is a species of interest under comparative analysis to the target species by the technology disclosed, wherein the target species and a particular non-target species are non-overlapping. In many implementations, the non-target species is selected to be an evolutionarily proximate species relative to the target species. However, a particular species need not be evolutionarily proximate to the target species to be selected as a non-target species and evolutionary distance to the selected target species should not be considered a limitation of the technology disclosed.

Herein, an evolutionarily proximate species may also be referred to synonymously as a closely-related species, orthologous species, or homologous species, wherein homology refers to similarity of the structure, physiology, or development of different species of organisms based on their shared evolutionary ancestry and orthology refers to a particular class of homology wherein homology is retained following speciation. Analogous to the homology of two or more species, genes may also be classified as homologous or orthologous. Herein, the terms “homologous genes”, “orthologous genes”, “homologs”, and “orthologs” may be used synonymously.

The technology disclosed generally relates to the comparison of target species and non-target species. In some implementations, the target species may be compared to a single non-target species. In other implementations, the target species may be compared to an aggregated cohort of two or more non-target species. A cohort of non-target species may be aggregated by a variety of methods (e.g., pooling values, averaging values, fitting a distribution of values, and many other descriptive statistics and functions) without deviating from the scope of the technology disclosed and as such, should not be considered a limitation. Frequently, the terms “cohort of non-target species”, “one or more non-target species”, “plurality of non-target species”, or simply “non-target species” are used interchangeably herein to describe a plurality of embodiments of the disclosed technology. Moreover, many implementations of one or more components of the disclosed technology are explained in the context of a plurality of non-target species for simplicity. Nonetheless, it is to be understood that the technology disclosed may be implemented for any number of non-target species (for example, calculations wherein non-target species data is pooled into a single cohort, a single non-target species forms a cohort of one).

In some embodiments, the technology disclosed further comprises a plurality of statistical models and probability distributions applied to the genetic data of species under analysis. A number of systems, algorithms, models, et cetera may be leveraged to the same end; however, in the interest of conciseness, a limited number of example models are described in detail herein as a user skilled in the art will recognize the ways in which the disclosed models may be altered or augmented in accordance with the technology disclosed. These models include, but are not limited to, population genetics models, regression models, classification models, significance tests, machine learning classifiers, or correction methods.

The statistical models disclosed within various implementations of the technology disclosed process data related to genes and genetic variation within genetic sequences. A particular gene is said to exist in a number of genotypes, or variations of the gene with non-identical sequences (also referred to as a variant). Genotypes may also be referred to synonymously as alleles. Sometimes within scientific literature, a narrow definition of an allele may be used to define a genotype comprises a pair of alleles. Herein, the broader definition of an allele is used such that “genotype”, “allele”, “variant”, and “genetic variant” are all used synonymously.

Variants are differentiated from one another by their respective mutations, or changes to the nucleic acid sequence within that gene or genomic region. For simplicity, this disclosure provides single nucleotide polymorphisms as an example of a type of mutation, but other classes of mutation exist. The terms “single nucleotide polymorphism”, “single nucleotide variant”, “segregating site”, “segregating variant”, “mutation”, or “segregating mutation” may all be used herein and should be understood as equivalent terminology. Mutations that do not change the protein generated by a particular gene are also referred to as synonymous mutations, whereas protein-changing mutations are referred to as nonsynonymous. For a position within a genetic sequence that does not have a known variation within a population, the position may be described as “fixed” or “conserved”, contrasted by “non-conserved” positions within a genetic sequence with known variation.

### System Overview: Explicit Population Genetic Model of Selection

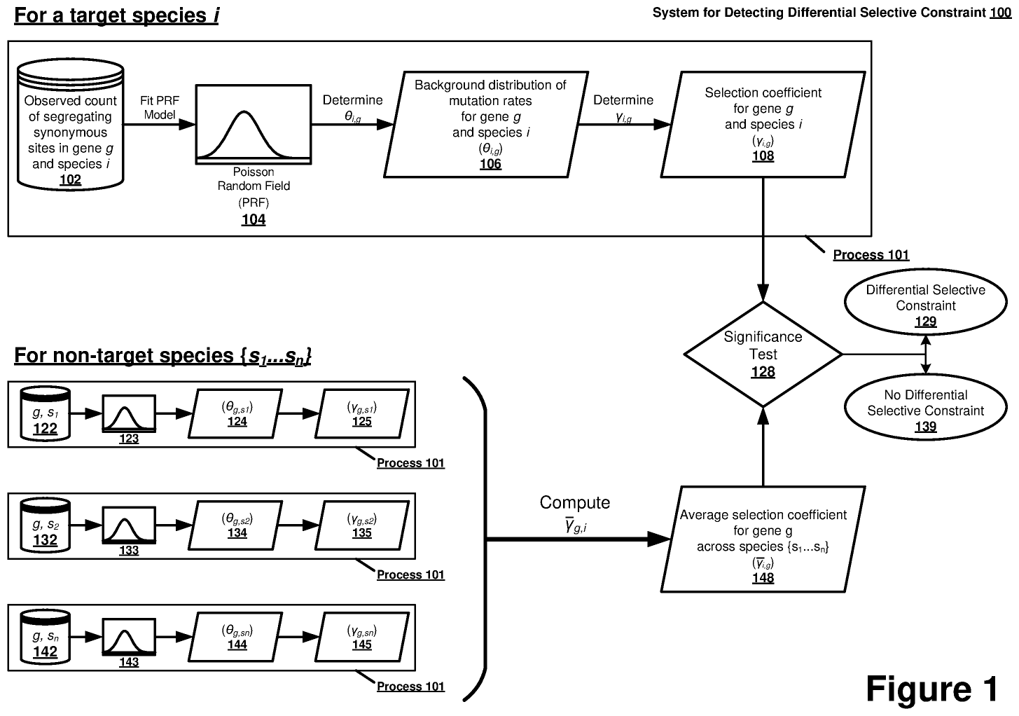

FIG. 1 is a schematic diagram of a system 100 for detecting differential constraint via the comparison of selection coefficients, according to one embodiment of the disclosed technology. The discussion of FIG. 1 is organized as follows. First, the elements of the figure are described, followed by their interconnections. Then, the use of the elements in the system is described in greater detail.

System 100 comprises a process 101 configured to obtain counts of segregating sites per gene per species and fit a Poisson Random Field (PRF) model 104 to the observed count of segregating sites, wherein the observed count of segregating sites is a Poisson random variable with a mean value determined by a mutation rate, a demography, and a sample size. The system 100 uses the PRF model 104 to learn a neutral background distribution of mutation rates per gene per species. The system 100 further applies the neutral background distribution of mutation rates per gene per species to estimate the average population-scaled selection coefficient per gene across species, wherein the average population-scaled selection coefficient per gene across species for a single species is equal to the estimated population-scaled selection coefficient per gene learned from the PRF model per gene, and wherein the average population-scaled selection coefficient per gene across species for two or more species is equal to the average value of the sum of each estimated population-scaled selection coefficient per gene per species learned from the PRF model per gene per species.

In some embodiments, the system further applies process 101 to a target species I to determine an observed count of segregating sites in gene g and species i from database 102. As part of the process 101, the system 100 also fits the PRF model 104 to the count of segregating sites in gene g and species i. In addition, the system 100 applies or employs the PRF model 104 to learn the background distribution of mutation rates for gene g and species i 106. Based on the background distribution of mutation rates for gene g and species i 106, the system 100 further estimates an average selection coefficient for gene g and species i 108.

Additionally, in some cases, the system 100 applies process 101 to a plurality of non-target species {s1 . . . sn} to determine an observed count of segregating sites in gene g and non-target species s1 from database 122. The system 100 fits the obtained observed counts of segregating sites in gene g and species s1 to PRF model 123 to the count of segregating sites in gene g and non-target species s1. The system 100 further applies or employs the PRF model 123 to learn the background distribution of mutation rates for gene g and non-target species s1 124. Based on the background distribution of mutation rates for gene g and non-target species s1 124, the system 100 further estimates a selection coefficient for gene g and non-target species s1 125. In a similar fashion, the system 100 applies the process 101 to non-target species s2 (wherein process 101 is followed as previously described, correspondingly to database 132, PRF model 133, background distribution 134, and selection coefficient 145) through non-target species sn (wherein process 101 is followed as previously described, correspondingly to database 142, PRF model 143, background distribution 144, and selection coefficient 145).

Next, the system 100 determines or computes the average selection coefficient for gene g across non-target species {s1 . . . sn} 148. For instance, the system 100 determines an average of the sum of each estimated selection coefficient for gene g per species (e.g., coefficients 125 through 145).

In some embodiments, the system 100 tests a pair of average population-scaled selection coefficients per gene across species, such as the selection coefficient for gene g in target species i 108 and the average selection coefficient for gene g across non-target species {s1 . . . sn} 148, for significance via significance test 128. Significance test 128 may be a likelihood-ratio test, a chi-squared test, a t-test, and so on. A user skilled in the art will recognize that the system 100 may implement a plurality of significance tests (with the appropriate data standardizations to meet necessary statistical assumptions) as significance test 128.

In applying the significance test 128, the system 100 can utilize a null model (or null hypothesis) such that the selection coefficient for gene g in target species i 108 and the average selection coefficient for gene g across non-target species {s1 . . . sn} 148 are not significantly different. Conversely, in some embodiments, the system 100 can utilize an alternative model (or alternative hypothesis) when applying the significance test 128 such that the selection coefficient for gene g in target species i 108 and the average selection coefficient for gene g across non-target species {s1 . . . sn} 148 are significantly different. For the significance test 128, significance is defined by a pre-determined alpha level. If the system 100 determines a difference between the selection coefficient for gene g in target species i 108 and the average selection coefficient for gene g across non-target species {s1 . . . sn} 148 to be significantly different (e.g., to satisfy a threshold alpha level), the system 100 can thus determine that there is differential selective constraint for gene g between target species i and non-target species {s1 . . . sn} 129. If the system 100 determines a difference between the selection coefficient for gene g in target species i 108 and the average selection coefficient for gene g across non-target species {s1 . . . sn} 148 to not be significantly different (e.g., to not satisfy a threshold alpha level), the system 100 can thus determine that there is no differential selective constraint for gene g between target species i and non-target species {s1 . . . sn} 139.

Further continuing with the description of system 100, the components of the system 100 illustrated in FIG. 1 can be implemented by software running on varying computing devices. Example computing devices for the system 100 include a workstation, a server, a computing cluster, a blade server, a server farm, or any other data processing system or computing device. The system 100 can also include a processing engine communicably coupled to one or more databases (e.g., the databases 102, 122, 132, and/or 142) via a network connection.

While system 100 is described herein with reference to particular blocks, it is to be understood that the blocks are defined for convenience of description and are not intended to require a particular physical arrangement of component parts. Further, the blocks need not correspond to physically distinct components. To the extent that physical distinct components are used, connections between components can be wired and/or wireless as desired. The different elements or components can be combined into single software modules and multiple software modules can run on the same hardware.

Prior to the discussion of the series of computations necessary to obtain an estimate of the neutral background distribution of mutation rates from a count of segregating sites and consequently obtain a selection coefficient for a particular gene, this disclosure describes the structure and implications of genetic variants, segregating sites, mutation, and selection.

Genetic Variation

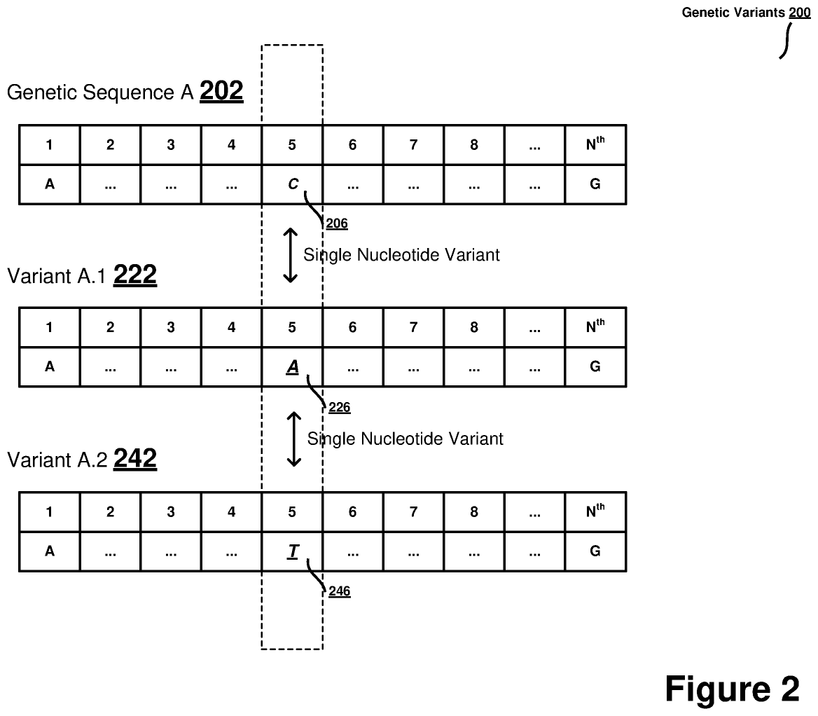

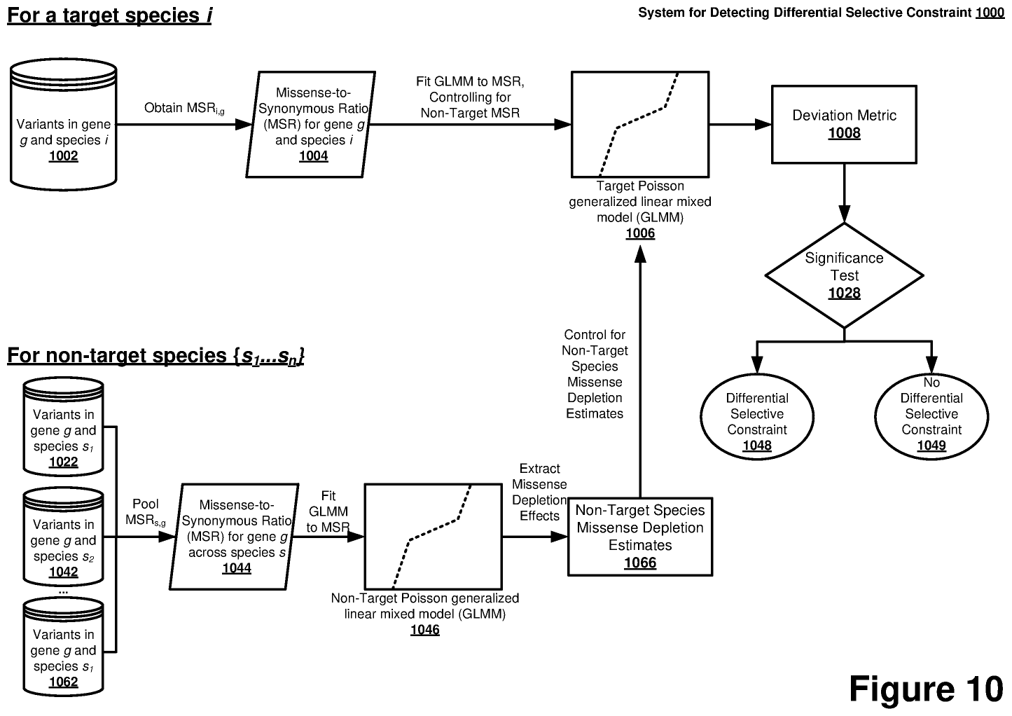

The disclosed methods and systems of predicting differential selective constraint on a gene-by-gene basis involves data associated with sequenced genomic data from individuals belonging to a particular species. The discussion now turns to details of genomic data, their associated features, and the relationship between genotype and phenotype for a particular individual. The following description of FIG. 2 through FIG. 8 refers to certain embodiments of the system 100. As indicated below, in some embodiments, a system 1000 for detecting differential constraint depicted in FIG. 10 can perform the same methods, determinations, operations, and functionalities described with reference to FIG. 2 through FIG. 8.

FIG. 2 shows an example genetic sequence 202 and corresponding variants resulting from a single nucleotide polymorphism. An example reference genetic sequence A 202 possesses a length of N wherein each sequence position comprises a nucleotide base of either thymine, adenine, guanine, or cytosine. The reference genetic sequence A 202 can be considered the wild type, or typical, genetic sequence for a segment of a particular gene. In some embodiments, the system 100 compares the reference genetic sequence A 202 with two example variant sequences A.1 222 and A.2 242, where each of the variant sequences possesses a respective single nucleotide variant in the fifth base position but otherwise possess an identical composition to the reference genetic sequence A 202. For example, at the fifth base position, the system 100 identifies a single nucleotide substitution as adenine 226 in variant A.1 222 and thymine 246 in variant A.2 242 as compared to cytosine 206 in the reference genetic sequence A 202. In some embodiments, the system 100 digitally transcribes and translates reference genetic sequence A 202, variant A.1 222, and variant A.2 242 into amino acid units of a protein. As a result of such digital transcription and translation, single nucleotide variants 226 and 246 may respectively result in at least one similar or different amino acid as compared to the amino acid(s) present in the wild type protein. If the system 100 determines that variant A.1 222 or variant A.2 242 respectively result in at least one changed amino acid in the translated protein, the system 100 may also determine that the respective variant also results in a changed phenotype for an individual possessing the respective variant.

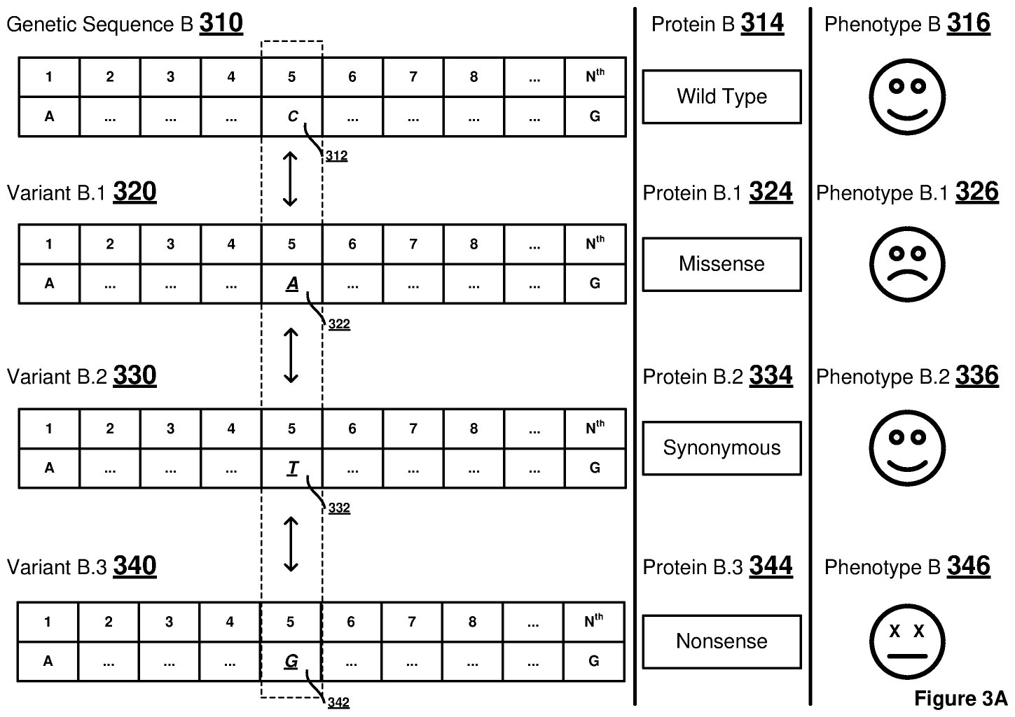

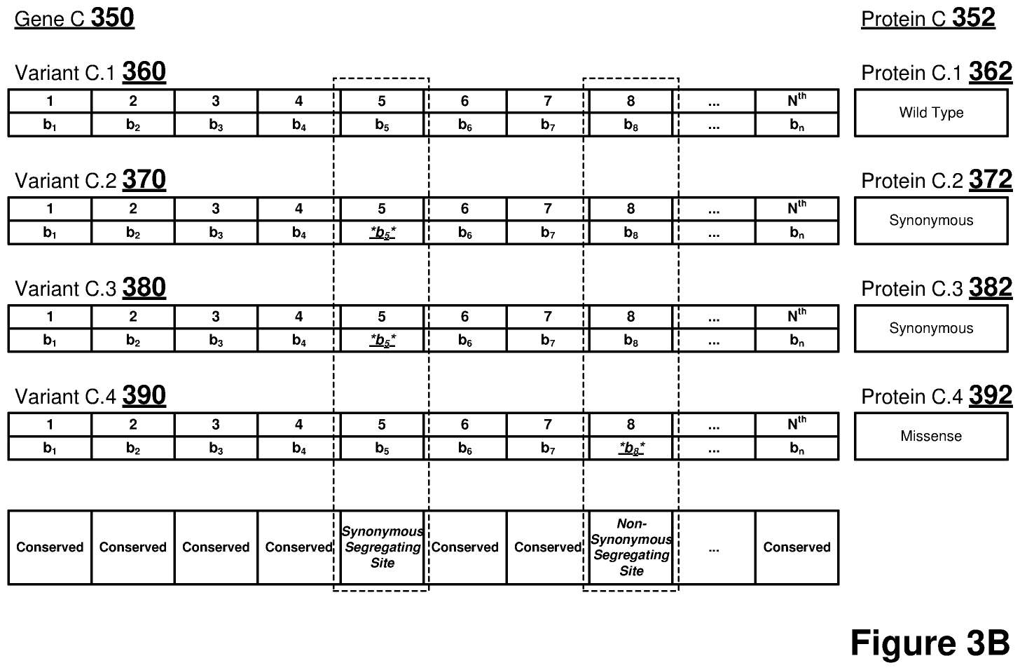

FIG. 3A shows an example genetic sequence 310 and corresponding variant effects resulting from a single nucleotide polymorphism. FIG. 3A is an example illustrating phenotypic effects in response to changes in genotype. An example reference genetic sequence B 310 possesses a length of N wherein each sequence position comprises a nucleotide base of either thymine, adenine, guanine, or cytosine. The reference genetic sequence B 310 can be considered the wild type, or typical, genetic sequence for a segment of a particular gene. In some embodiments, the system 100 compares the reference genetic sequence B 310 with three example variant sequences B.1 320, B.2 330, and B.3 340, where each of the variant sequences possesses a respective single nucleotide variant in the fifth base position but otherwise possess an identical composition to the reference sequence. For example, the system 100 identifies or determines, at the fifth base position, a single nucleotide substitution as adenine 322 in variant B.1 320, thymine 332 in variant B.2 330, and guanine 342 in variant B.3 340, when comparing to cytosine 312 in the reference genetic sequence B 310.

The system 100 can digitally transcribe and translate reference genetic sequence B 310 into protein B 314, the wild type protein composition for the particular gene comprising reference genetic sequence B 310. In some cases, individuals possessing wild type protein B 314 will present with phenotype B 316, a healthy phenotype. The system 100 further determines that variant B.1 320 results in mutant protein B.1 324, where mutant protein B.1 324 comprises a missense mutation resulting in a differing protein structure and function from the wild type protein B 314. In some cases, individuals possessing missense protein B.1 324 will present with phenotype B.1 326, a disease phenotype. In addition, the system 100 determines that variant B.2 330 results in mutant protein B.2 334, where mutant protein B.2 334 comprises a synonymous mutation resulting in no change to protein structure and function as compared to the wild type protein B 314 (i.e., no amino acid changes occurred in response to the single nucleotide variant in position five of variant B.2 330). In some cases, individuals possessing synonymous protein B.2 334 will present with phenotype B.2 336, a healthy phenotype similar to phenotype B 316. Further, the system 100 determines that variant B.3 340 results in mutant protein B.3 344, where mutant protein B.3 344 comprises a nonsense mutation resulting in a truncated, nonfunctional protein structure and function as compared to wild type protein B 314. In some cases, individuals possessing nonsense protein B.3 344 will present with phenotype B.3 346, likely resulting in a nonviable embryo or significantly reduced lifespan.

A person skilled in the art will recognize that variants B.1 320, B.2 330, and B.3 340 are listed as simplified examples and potential phenotypic representations span a wide spectrum rather than a limited number of discrete representations. Moreover, a person skilled in the art will also recognize that many phenotypic responses occur due a plurality of variants within a single gene, or a combined polygenic variant effect. Many implementations of the technology disclosed specifically address polygenic risk scoring for a particular phenotypic response associated with severe genetic disorders known to cause substantial detriment to an individual's quality of life and/or life expectancy.

FIG. 3B contrasts conserved positions with segregating sites within a group of allelic variants. An example reference gene C 350 possesses a length of N wherein each sequence position bn comprises a nucleotide base of either thymine, adenine, guanine, or cytosine. The reference gene C 350 can exist as the wild type, or typical, genetic sequence, variant C.1 360. The system 100 determines that variant C.1 360 results in the wild type phenotype of protein C 352, protein C.1 362. In addition, the system 100 determines that the reference genetic sequence C 350 also exists as additional variants C.2 370, C.3 380, and C.4 390. Analyzing variant C.2 370, the system 100 determines that variant C.2 370 comprises a single nucleotide polymorphism in position b5 and results in synonymous protein C.2 372. In some cases, the system 100 determines that a protein variant for protein 352 that is determined to be synonymous to the wild type does not have a change to the protein structure or function, relative to wild type protein C.1 362.

Additionally, the system 100 determines that variant C.3 380 also comprises a single nucleotide polymorphism in position b5 and results in synonymous protein C.3 382. Although Variant C.3 380 has a single nucleotide polymorphism in the same position as Variant C.2 370, the system 100 classifies them as separate variants because the substituted nucleic acid base is not the same. However, because both protein C.3 382 and protein C.2 372 are both synonymous to wild-type, the system 100 also determines that b5 is in a “wobble” position within a codon that is translated into an amino acid within protein C 352. In some cases, the wobble effect refers to the redundancy within the genetic code that allows for a certain degree of variation within the third position of a codon that will not affect the amino acid that will be translated from the codon.

As further illustrated in FIG. 3B, the system 100 determines that variant C.4 390 comprises a single nucleotide polymorphism in position b8 and results in a missense mutation within protein C.4 392. As used herein, missense mutations and nonsense mutations are both classified as nonsynonymous mutations. The system 100 can thus determine that a protein variant for protein C 352 that is nonsynonymous to the wild type comprises at least one change to the protein structure or function, relative to wild type protein C.1 362.

Continuing the discussion of FIG. 3B, the system 100 can determine that all positions other than b5 and b8 are conserved (or fixed sites) in the alignment of the four variants C.1 360, C.2 370, C.3 380, and C.4 390 for gene C 350. The system 100 can also determine that position b5 is a synonymous segregating site, as both known variants are not observed to cause any amino acid changes within the resulting composition of protein C 352. In contrast, the system 100 can determine that position b8 is a nonsynonymous segregating site, as variant C.4 390 comprising a mutation at site b8 results in a change to the amino acid composition of protein C 352.

As further illustrated in FIG. 3B, the system 100 can identify or determine that the frequencies of each variant for gene C 350 are influenced by a number of factors, including environmental change, demographic change, random mutation, and natural selection. In some embodiments, the system 100 determines a natural selection effect onto the allelic frequencies of gene C 350. Based on such a natural selection effect, the system 100 can determine the origin and/or adaptive significance of a particular variant due to the relationship between natural selection and the relative fitness of the particular variant. As set forth further below, in some embodiments, the system 100 can determine and quantify such a natural-selection relationship and relative fitness by determining a selection coefficient and/or an indicator of differential selective constraint.

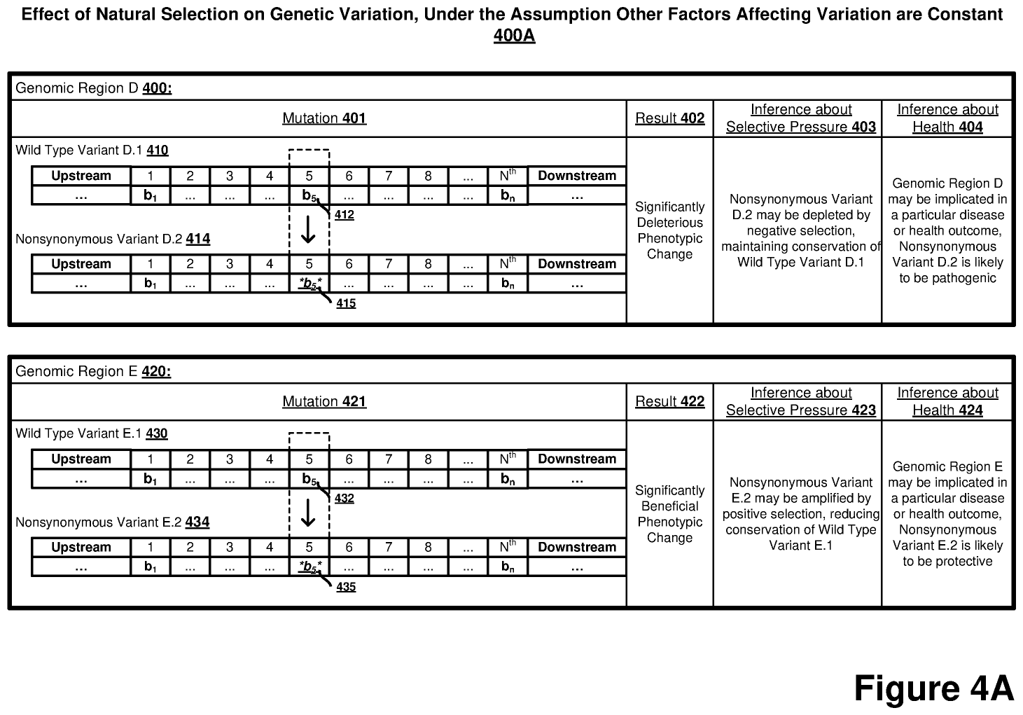

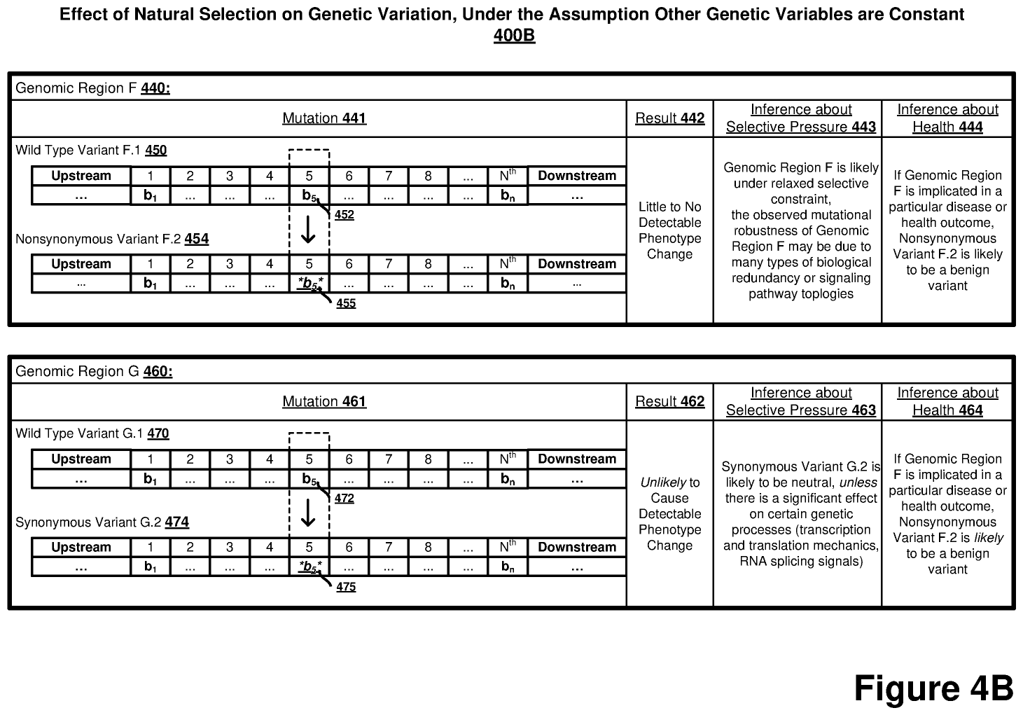

FIG. 4A shows a plurality of sample scenarios 400A illustrating the relationship between natural selection and genetic variation, wherein the examples exhibit evidence for greater selective constraint. Although unrealistic in true populations, for the sake of focusing exclusively on natural selection, the discussion of FIG. 4A (and other figures) assumes that other factors effecting genetic variation are constant (e.g., no significant change to population structure, environmental factors, or mutation rates). Generally speaking, genetic variants with higher relative fitness are expected to have a greater relative contribution to the overall reproductive success of an organism or a population within a given species than alternate variants. In a simple gene with two variants, if organisms expressing the first of two variants on average produce more offspring or survive longer than organisms expressing the second of two variants, the system 100 determines first variant to have a higher relative fitness to the second variant.

As illustrated in FIG. 4A, genomic region D 400 possesses a length of N wherein each sequence position bn comprises a nucleotide base of either thymine, adenine, guanine, or cytosine. Genomic region D 400 can exist as the wild type, or typical, genetic sequence, variant D.1 410. In the event that mutation 401 occurs at position b5, the system 100 determines that nonsynonymous variant D.2 414 results with substituted base 415 in place of base 412. The system 100 further determines that nonsynonymous variant D.2 414 results in a phenotypic change 412 that is significantly deleterious to the health of organisms expressing nonsynonymous variant D.2 414. Under an established scenario with the given set of assumptions, the system 100 determines (or infers) the selective pressure 403 that will affect the frequency of variants within genomic region D 400. In some cases, organisms expressing nonsynonymous variant D.2 414 have a lower relative fitness than wild type variant D.1 410. Accordingly, the system 100 determines (or estimates) that negative selection (also known as purifying selection) will deplete the frequency of nonsynonymous variant D.2 414 within a population to maintain conservation of wild type variant D.1 410. Assuming that wild type variant D.1 410 has the highest relative fitness of all variants for genomic region D 400 within a population, the system 100 can also determine or estimate that there is selective constraint acting upon genomic region D 400 to conserve wild type variant D.1 410.

The system 100 can further make determinations (or inferences) about the health impact 404 of mutation 401 in genomic region D 400. For example, the system 100 determines, detects, or identifies a lack of tolerance for mutation 401 from wild type variant D.1 410 to nonsynonymous variant D.2 414. As a result of the lack of tolerance, the system 100 can determine that genomic region D 400 is implicated in a particular disease or health outcome. In some embodiments, the system 100 can determine or estimate that nonsynonymous variant D.2 414 is a pathogenic variant for genomic region D 400.

While the discussion of genetic variation in the context of modern medicine often focuses on the deleterious effect of pathogenic variants versus benign variants, beneficial variants also exist in the broader context of evolutionary processes.

As further illustrated in FIG. 4A, genomic region E 420 possesses a length of N wherein each sequence position bn comprises a nucleotide base of either thymine, adenine, guanine, or cytosine. Genomic region E 420 can exist as the wild type, or typical, genetic sequence, variant E.1 430. In the event that the system 100 detects, identifies, or determines a mutation 421 at position b5, the system 100 further determines that nonsynonymous variant E.2 434 results with substituted base 435 in place of base 432. The system 100 further determines or detects that nonsynonymous variant E.2 434 results in a phenotypic change 422 that is significantly beneficial to the health of organisms expressing nonsynonymous variant E.2 434. Under the established scenario with the given set of assumptions described herein, the system 100 can determine (or infer) the selective pressure 423 that will affect the frequency of variants within genomic region E 420. In some embodiments, organisms expressing nonsynonymous variant E.2 434 have a higher relative fitness than wild type variant E.1 430. Accordingly, the system 100 can determine or estimate that positive selection (also known as adaptive selection) will amplify the frequency of nonsynonymous variant E.2 434 within a population and reduce the frequency of wild type variant E.1 430. Given that nonsynonymous variant E.2 434 is tolerated well within genomic region E 400 within a population, the system 100 can also determine or estimate that greater selective constraint acting upon genomic region E 400 to amplify the frequency of nonsynonymous variant E.2 434.

The system 100 can further make inferences about the health impact 424 of mutation 421 in genomic region E 420. The advantage gained via mutation 421 from wild type variant E.1 430 to nonsynonymous variant E.2 434 suggests that if genomic region E 420 is implicated in a particular disease or health outcome, the system 100 can determine or estimate that nonsynonymous variant E.2 434 is a protective, beneficial variant for genomic region E 420.

FIG. 4B shows a plurality of sample scenarios 400B illustrating the relationship between natural selection and genetic variation, wherein the examples exhibit evidence for relaxed selective constraint. Genomic region F 440 possesses a length of N wherein each sequence position bn comprises a nucleotide base of either thymine, adenine, guanine, or cytosine. Genomic region F 440 can exist as the wild type, or typical, genetic sequence, variant F.1 450. In the event that mutation 441 occurs at position b5, the system 100 determines or detects that nonsynonymous variant F.2 454 results with substituted base 455 in place of base 452. The system 100 further determines or detects that nonsynonymous variant F.2 454 results in a phenotypic change 442 that has little to no detectable phenotype change within organisms expressing nonsynonymous variant F.2 454. Under the established scenario with the given set of assumptions described herein, the system 100 can determine (or infer) that genomic region F 440 is likely under more relaxed selective constraint 443. In some cases, nonsynonymous variant F.2 454 has a comparable relative fitness to wild type variant F.1 450. Accordingly, the system 100 can determines or estimates that genomic region F 400 has a higher degree of mutational robustness, which may have resulted from compensation by another biological pathway or gene (i.e., biological redundancy).

In some embodiments, the system 100 can further make inferences about the health impact 444 of mutation 441 in genomic region F 440. In some cases, the lack of phenotype change resulting from mutation 441 from wild type variant F.1 450 to nonsynonymous variant F.2 454 makes it difficult to determine or estimate if genomic region F 440 is implicated in a particular disease or health outcome without further information. In the event that genomic region F 440 is shown to be implicated in a particular disease or health outcome, the system 100 can determine or estimate that nonsynonymous variant F.2 454 is a benign variant for genomic region F 440.

As further illustrated in FIG. 4B, genomic region G 460 possesses a length of N wherein each sequence position bn comprises a nucleotide base of either thymine, adenine, guanine, or cytosine. Genomic region G 460 can exist as the wild type, or typical, genetic sequence, variant G.1 470. In the event that mutation 461 occurs at position b5, the system 100 can determine that synonymous variant G.2 474 results with substituted base 475 in place of base 472. As shown, the system 100 determines that it is unlikely that synonymous variant G.2 474 will result in a phenotypic change 462. While it has long been assumed in the field that synonymous substitutions are neutral substitutions that are not subjected to natural selection, recent genomics research suggests that even synonymous variants may be affected by varying magnitudes of selective forces as a result of changes within codon bias, RNA splicing, and other genetic machinery and processes. However, more research is necessary to understand the extent to which this phenomenon occurs, thus, it is reasonable for the system 100 to determine that synonymous variant G.2 474 is unlikely to cause detectable phenotypic change, but causing detectable phenotypic change is possible. Under the established scenario with the given set of assumptions described herein, in some cases, existing systems cannot determine or estimate the selective pressure exerted onto genomic region G 460 without further information concerning genetic processes 463.

Existing systems are similarly limited in their ability to make inferences about the health impact 464 of mutation 461 in genomic region G 460. In the event that genomic region G 460 is shown to be implicated in a particular disease or health outcome, the system 100 can estimate that synonymous variant G.2 474 is likely to be a benign variant for genomic region G 460. However, newly-developing research is changing the paradigm for the impact of synonymous variation and suggests that certain genomic diseases, such as cystic fibrosis, may be associated with splicing dysfunction caused by variants that otherwise do not change the amino acid sequence of a resulting protein.

Concepts within evolutionary genetics, such as fitness and selective pressure, can also be quantified, and this quantification is a substantial component of the analysis performed within evolutionary medicine technologies.

Relative Fitness and Selection Coefficient as Measures of Selective Constraint

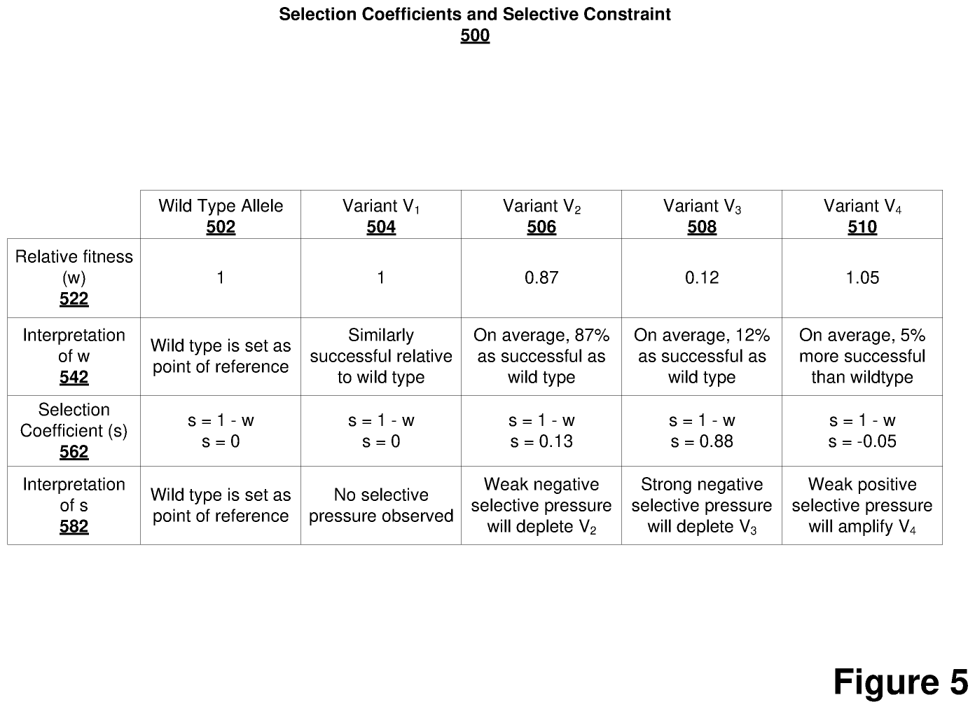

FIG. 5 shows the relationship between relative fitness and natural selection for an example gene. As illustrated, the example gene is observed as a wild type allele 502, variant V1 504, variant V2 506, variant V3 508, and variant V4 510. The relative fitness of a particular allele within a gene can be expressed as the ratio of fitness of the particular allele to the fitness of the allele with the highest relative fitness within the genetic population. In some embodiments, the system 100 determines or calculates relative fitness relative to the wild type allele by default because the wild type allele should almost always be the allele with the highest relative fitness within the genetic population. Thus, the system 100 sets relative fitness 522 of wild type allele 502 as the point of reference and considers it equivalent to one.

As further shown in FIG. 5, the system 100 can determine, via analysis of reproductive rates within the genetic population, that the relative fitness of variant V1 504 is equal to 1, the relative fitness of variant V2 506 is equal to 0.87, the relative fitness of variant V3 508 is equal to 0.12, and the relative fitness of variant V4 510 Is equal to 1.05. Additionally, the system 100 can determine or interpret the relative fitness values 542 to mean that on average, variant V1 504 is similarly successful to the wild type variant 502, variant V2 506 is 87% as successful compared to the wild type variant 502, variant V3 508 is 12% as successful compared to the wild type variant 502, and variant V4 510 is 5% more successful than wild type variant 502.

The system 100 can further apply the relative fitness of each variant to determine the selection coefficient 562 of each variant, where the selection coefficient 562 is equal to 1 minus the relative fitness of a particular variant. In some cases, the selection coefficient of a particular variant for a particular gene is a measure of differences in relative fitness 582. Thus, given the relative fitness values, the system 100 can determine or compute that the selection coefficient of variant V1 504 is equal to 0, the selection coefficient of variant V2 506 is equal to 0.13, the selection coefficient of variant V3 508 is equal to 0.88, and the selection coefficient of variant V4 510 is equal to −0.05. Because the system 100 set the wild type allele 502 as the point of reference, the system 100 determines that the selection coefficient is 0, thus identifying that there is no selective pressure acting on wild type allele 502. Similarly, because the selection coefficient of variant V1 504 is also equal to 0, the system 100 can determine or infer that no selective pressure is acting on variant V1 504.

As shown, positive selective pressure is acting on both variant V2 506 and variant V3 508, but the system 100 determines, based on the selection coefficients, that the negative selective pressure acting on variant V3 508 is stronger and will deplete the frequency of variant V3 508 within the genetic population faster than that of variant V2 506. In contrast, the system 100 determines, based on the selection coefficient of variant V4 510, that a weak positive selective pressure will gradually amplify the frequency of variant V4 510 within the genetic population.

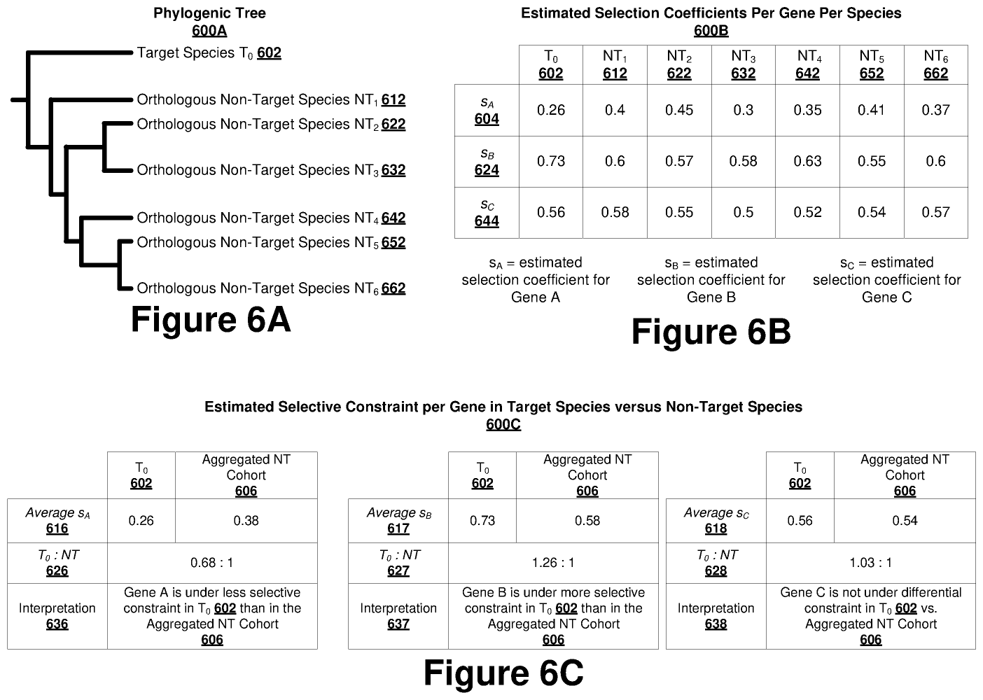

To determine the differential selective constraint acting on a gene between homologous species, the system 100 can determine or approximate a single selection coefficient value for all variants within the particular gene per species. The system 100 can further compare the selection coefficients to identify the extent of differential selective constraint between species. FIGS. 6A through 6C demonstrate this process.

FIG. 6A shows a phylogenic tree 600A for a target species and non-target species orthologous to the target species. Target species TO 602 is evolutionarily proximate to a plurality of orthologous non-target species NT1 612, NT2 622, NT3 632, NT4 642, NT5 652, and NT6 662.

FIG. 6B shows representative selection coefficients per gene per species. For each species TO 602, NT1 612, NT2 622, NT3 632, NT4 642, NT5 652, and NT6 662, the system 100 identifies a single estimated selection coefficient per gene per species for a gene A 604, gene B 624, and gene C 644.

FIG. 6C shows representative sample calculations of selective constraint per gene using selection coefficients as measures for selection. First, the system 100 defines an aggregated NT cohort 606, and averages the estimated selection coefficients per gene per species across different species. Accordingly, the system 100 determines or obtains a single average estimated selection coefficient per gene across species such that the aggregated NT cohort 606 is able to share genetic data across species to control for outlier values. The system 100 determines or identifies the average estimated selection coefficients per gene across species for gene A individually in row 616 and determines or computes a ratio of the selection coefficients 626 to be 0.68:1. The system 100 further analyzes or interprets the ratio 626 to suggest that Gene A is under less selective constraint in T0 602 than in the Aggregated NT Cohort 606. Likewise, the system 100 determines or identifies the average estimated selection coefficients per gene across species for gene B individually in row 617 and determines or computes a ratio of the selection coefficients 627 to be 1.26:1. The system 100 further analyzes or interprets the ratio 627 to suggest that Gene B is under more selective constraint in T0 602 than in the Aggregated NT Cohort 606. Finally, the system 100 determines or identifies the average estimated selection coefficients per gene across species for gene C individually in row 618 and determines or computes a ratio of the selection coefficients 628 to be 1.03:1. In addition, the system 100 analyzes or interprets the ratio 628 to suggest that Gene B is not under differential selective constraint in T0 602 than in the Aggregated NT Cohort 606 due to the similarity of the values.

The above examples in FIGS. 2 through 6C are simplified to focus on the conceptual framework underlying the relationships leveraged in the technology disclosed, wherein a user skilled in the art will recognize that while the number of variants, reported values, and required assumptions are unrealistic for real genetic populations, the concepts demonstrated are valid. The discussion now turns to the description of a population genetic model to estimate mutation rates per gene per species and the use of estimated selection coefficients to identify genes with differential selective constraint between species for real-world data obtained in humans and non-human primates. Because genomic populations are shaped in complex ways by sampling and demographic forces, sophisticated computations are needed.

Some implementations of the system 100 comprise an explicit population genetic model configured to further perform operations as part of a method that includes two phases: First, modelling the counts of synonymous segregating sites to learn a neutral background distribution of mutation rates per gene per species. Second, applying that neutral background distribution to estimate the average selection per gene across species.

Population Genetic Model to Estimate Mutation Rates Per Gene Per Species

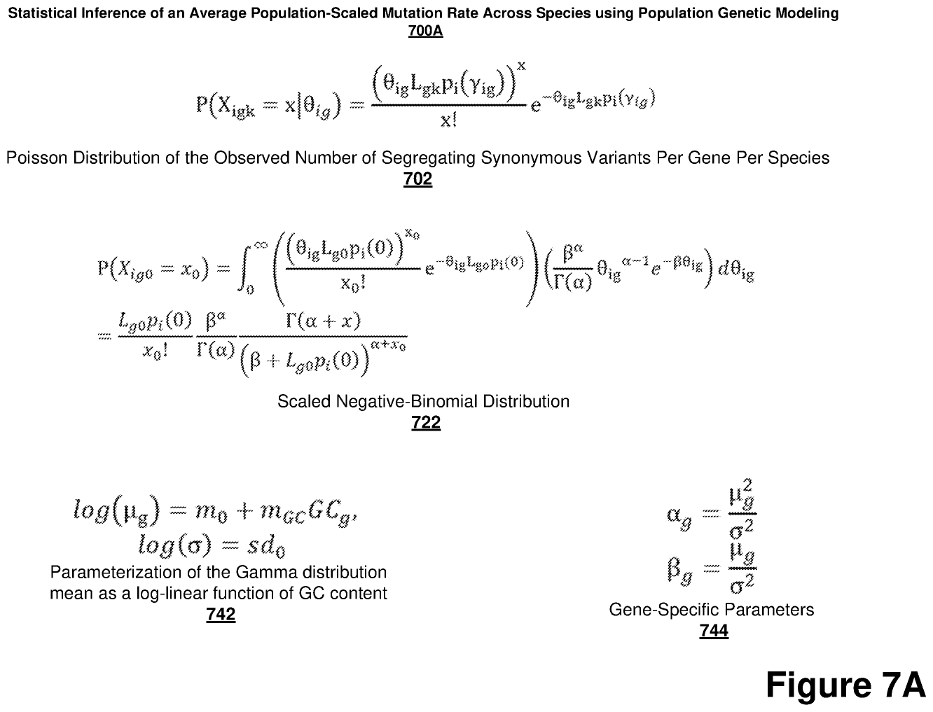

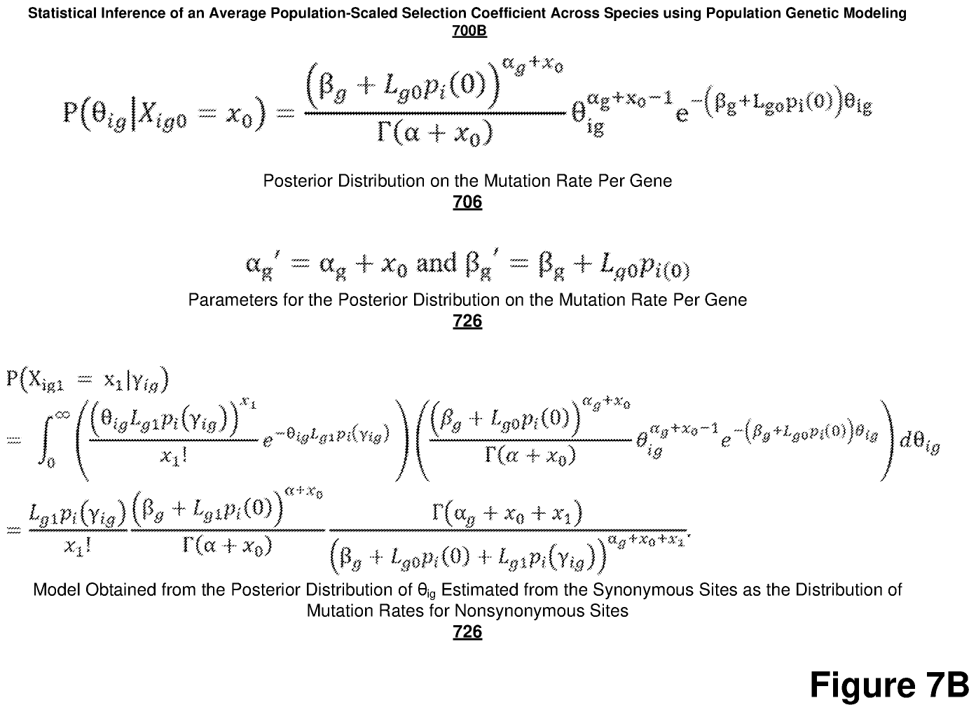

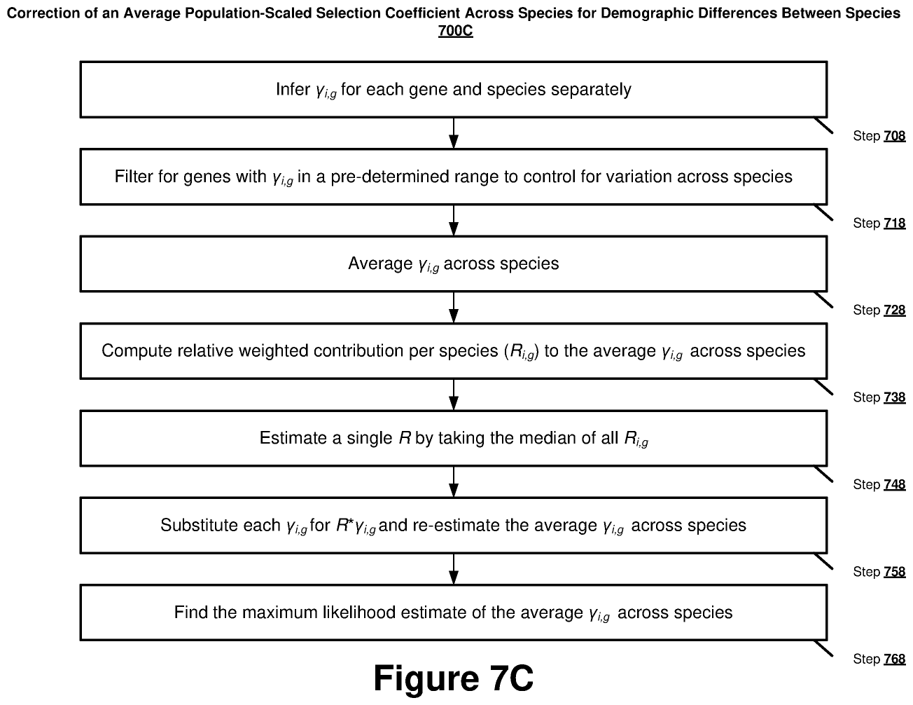

FIG. 7A shows a set of mathematical functions that the system 100 uses to estimate an average population-scaled mutation rate across species. While the process described within FIG. 7A through FIG. 7C is described in detail, it is to be understood that these details are given as a representation of one implementation of the technology disclosed.

As shown in FIG. 7A, the system 100 can perform multiple steps or operations to estimate an average population-scaled mutation rate across species. First, the system 100 can establish a neutral baseline for each species by fitting a model to the segregating synonymous variants in each species. Next, the system 100 can fit a Poisson Random Field (PRF) model where the observed number of segregating sites is a Poisson random variable. The system 100 can determine the mean for the Poisson random variable (or the PRF model) by mutation, demography, selection, and/or sample size. For simplicity, the system 100 can identify or assume an equilibrium (i.e., constant) demography for all species besides human. For human species, on the other hand, the system 100 can use biostatistical software to find a best fitting demographic history based on the folded site frequency spectrum of synonymous sites.

As shown, the system 100 determines or generates a best fitting demographic model where X_{iygk} is the number of mutations of type k (k=0 is synonymous, k=1 is missense) in gene g of species i, and where θ_{ig}=4Nᵢμ_{g} is the per site population scaled mutation rate, and further where L_{gk} is the number of sites of type k in gene g. Next, the system 100 can determine or compute pᵢ(γ_{ig}). Relating to pᵢ(γ_{ig}), in some cases, the system 100 determines θ_{ig}pᵢ(γ) as approximately the probability that a site in gene g with population scaled selection coefficient γ_{ig}=2Nᵢs_{g} is segregating in a sample from species i.

Accordingly, the system 100 determines the distribution of Xᵢⱼₖ to be Poisson with mean θ_{ig}L_{gk}pᵢ(γ_{ig}), such that the Poisson distribution of the observed number of segregating synonymous variants per gene per species is as shown in distribution 702.

In certain embodiments, due to a combination of true variation in mutation rate and data quality across the genome, the system 100 determines that different genes have a different effective per base-pair mutation rate. The system 100 accordingly implements or utilizes strategies to generate or create a robust estimate with few parameters per species. To accommodate this, the system 100 adopts a Gamma distributed prior on θ_{ig}, and applies the Gamma distributed prior to synonymous sites (i.e., k=0, γ_{ig}=0) and further integrates over the Gamma distributed prior to result in a scaled negative-binomial distribution 722.

As further illustrated in FIG. 7A, to account for the impact of GC content in mutation rate, the system 100 parameterizes the gamma distribution mean as a log-linear function of GC content, function 742. The system 100 can further determine or compute f gene-specific parameters 744 from the function 742.

Modeling Selection Across Species

FIG. 7B shows a set of mathematical functions that the system 100 uses to estimate an average population-scaled selection coefficient across species. Given the optimized parameters obtainable from the process detailed within the description of FIG. 7A, in some embodiments, the system 100 can compute a posterior distribution on the mutation rate per gene 706. Specifically, as shown in FIG. 7B, the system 100 determines the posterior distribution as a Gamma distribution with parameters 726.

As further shown in FIG. 7B, the system 100 can leverage or apply parameters m, m_{GC}, and sd₀ to parameterize the distribution of the number of segregating nonsynonymous sites given a selection coefficient. To generate the expected number of segregating sites given a selection coefficient, the system 100 can use simulation methods to generate pᵢ(γ) across a grid of population scaled selection coefficients, from 2Ns=0 to 2Ns=10000. In some cases, the system 100 determines that every nonsynonymous mutation in a gene shares the same population scaled selection coefficient, γ_{ig}. Additionally, the system 100 determines or estimates the posterior distribution of Big from the synonymous sites as the distribution of mutation rates for nonsynonymous sites and further applies the posterior distribution of θ_{ig} to obtain distribution 726.

Estimation of Average Selection Coefficients Per Gene

FIG. 7C shows a flow diagram comprising steps that the system 100 performs to correct an average population-scaled selection coefficient across species for demographic differences between species. Accordingly, in some cases, the system 100 determines from the processes described in FIGS. 7A and 7B the population-scaled selection coefficient, γ_{ig}=2Nᵢs_{ig}. Accordingly, the system 100 uses a multi-step procedure to control for demographic differences across species (i.e. differences in Nᵢ). In principle, the system 100 can determine γ_{ig} to be different across species due to differences in either Nᵢ or s_{ig}. In some embodiments, the system 100 determines that, for the majority of genes, s_{ig}≡s_{g} is identical across species, and further determines that the differences in γ_{ig} are driven primarily by differences in Nᵢ. Consequently, in these or other embodiments, the disclosed procedure that the system 100 performs or applies is analogous to an EM algorithm where the system 100 first determines or estimates γ_{ig} separately for each species and gene. As part of the procedure, the system 100 subsequently estimates Nᵢ by assuming that s_{ig} is identical across species, and finally applies the estimated Nᵢ to produce estimates of s_{g}.

As just mentioned, and as illustrated in FIG. 7C, the system 100 can perform a multi-step procedure to correct an average population-scaled selection coefficient across species for demographic differences between species. First, as shown in FIG. 7C, the system 100 determines or infers γ_{ig} for each gene and species separately in step 708. This may result in some information loss, because in species with small sample sizes, there may be many genes with 0 segregating missense variants, and hence result in an inference of an MLE γ_{ig}=−∞. To control for some variation in estimated γ across species, the system 100 performs step 718 to apply further restrictions to filter for genes with γ_{ig} between a pre-determined range of values. The pre-determined range of values may be species specific, gene specific, or allele frequency specific.

In some embodiments, the system 100 determines the difference in estimated γ_{ig}=2Nᵢsi_{g} between species to be due to the Nᵢ being different. In these or other embodiments, the system 100 determines that s_{ig}≡s_{g} is identical across species for gene g. For each gene, the system 100 performs step 728 to estimate γ_{ig} across species to obtain γ̅_̅{̅g̅}̅≈2N̅s_{g}, which represents the average population scaled selection coefficient for gene g. Additionally, the system 100 performs step 738 to determine or compute R_{ig}=γ_{ig}/γ̅_̅{̅g̅}̅, which is an estimator of Nᵢ/N̅ because γ_{ig}/γ_{g} 2Nᵢs_{g}/2N̅s_{g}=Nᵢ/N̅. To account for noise among the outliers and the fact that s_{g} is not truly identical across species, the system 100 performs step 748 to determine or estimate a single Rᵢ value per species by taking the median of R_{ig} across all genes in species i.

As further illustrated in FIG. 7C, the system 100 performs step 758 to aggregate species together to re-estimate γ̅_{g} by substituting γ_{ig}→Rᵢγ̅_{g}, which provides an approximation of γ_{ig} from γ̅_{g} via Rᵢγ̅_{g}≈Nᵢ/N̅2N̅s_{g}=²Nᵢs_{g}. Finally, as shown in FIG. 7C, the system 100 performs step 768 to increase or maximize the likelihood L(γ̅_{g})=Π_{i∈I}P(X_{ig1}|Rᵢγ̅_{g}) to find the maximum likelihood estimate of γ̅_̅{̅g̅}̅ across a group of species I. In certain implementations, the system 100 can set I to be either the set P of all non-human primates, or I to be the singleton set H containing just humans.

Identifying Differential Selective Constraint Between Target and Non-Target Species Leveraging Selection Coefficients

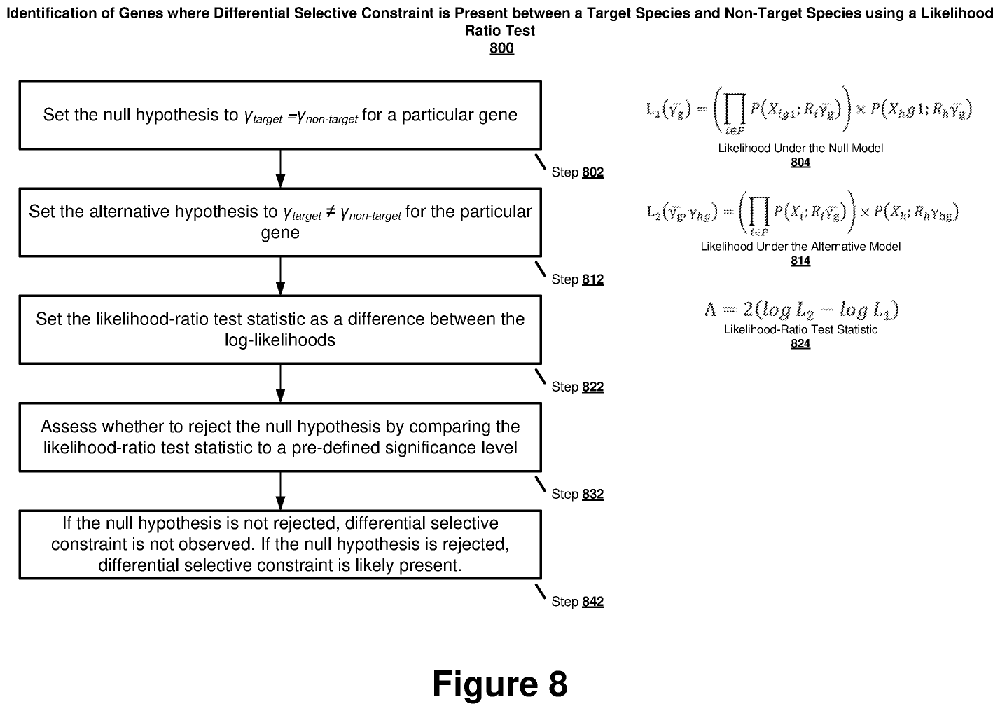

FIG. 8 shows a flow diagram comprising steps that the system 100 performs to identify genes where differential selective constraint is present between a target species and one or more non-target species using a likelihood-ratio test. To identify genes where human constraint is different from non-human primate selection, the system 100 performs the steps of a likelihood ratio test illustrated within the diagram 800. As shown, the system 100 performs the likelihood ratio test shown within the diagram 800 by testing whether s is significantly different between human and other primates that determines if γ̅_̅{̅g̅}̅ is different between human and primate. Under the null model illustrated in step 802, the system 100 determines γ_{gh}=γ̅_̅{̅g̅}̅. Based on this determination, the system 100 further determines the likelihood to be equivalent to the likelihood function 804 where P is the set of non-human primates and the subscript h indicates human data.

In some embodiments, the system 100 utilizes the alternative model illustrated in step 812 to determine γ_{gh}≠γ̅_̅{̅g̅}̅. Based on this determination, the system 100 further determines that the likelihood is equivalent to the likelihood function 814 where P is the set of non-human primates and the subscript h indicates human data.

As further illustrated in FIG. 8, the system 100 performs step 822 to set a likelihood ratio test statistic as the difference between the log-likelihoods. For instance, the system 100 determines the likelihood-ratio test statistic 824. Note that under the null model γ_{hg}=γ̅_̅{̅g̅}̅, the system 100 determines that L₁=L₂, which represents a nested model test (or a nested hypothesis test). Thus, under the null model that γₕ=γ̅, the system 100 determines that the test statistic (e.g., the likelihood-ratio test statistic 824) follows a X² distribution with one degree of freedom, Λ˜χ²(df=1), by standard likelihood ratio theory. Accordingly, the system 100 determines whether sₕg is significantly different from s_{g}, because correction has been performed for the effective population size of humans using Rₕ. Specifically, the system 100 determines that Rₕγ_{hg}≈Nₕ/N̅2N̅s_{hg}=2Nₕs_{hg} while Rₕγ̅_̅{̅g̅}̅ ≈Nₕ/N̅2N̅s_{g}=2N̅ₕs_{g}, so that if γ_{hg}≠γ̅_̅{̅g̅}̅, then s_{hg}≠s_{g}.

Computer System for Implementation of an Explicit Population Genetic Model of Selection

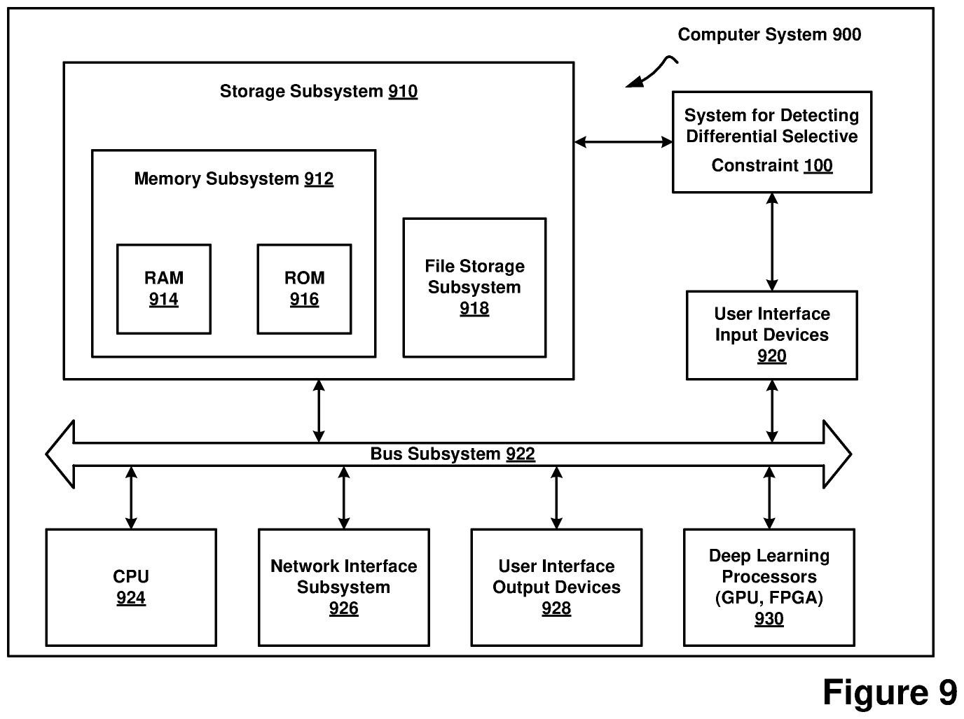

FIG. 9 shows a first example computer system (e.g., for the system 100) that can be used to implement the technology disclosed, according to one embodiment of the disclosed technology. Computer system 900 can facilitate or perform the actions or operations described herein relating to the system 100 (e.g., the system for detecting differential selective constraint). Indeed, as shown, the computer system 900 can house or store the system 100. As further shown, the computer system 900 includes at least one central processing unit (CPU) 924 that communicates with (or performs functions of) the system 100 and a number of peripheral devices via bus subsystem 922. These peripheral devices can include a storage subsystem 910 including, for example, memory devices and a file storage subsystem 918, user interface input devices 920, user interface output devices 928, and a network interface subsystem 926. The input and output devices allow user interaction with computer system 900. Network interface subsystem 926 provides an interface to outside networks, including an interface to corresponding interface devices in other computer systems.

In one implementation, the system 100 is communicably linked to the storage subsystem 910 and the user interface input devices 920 (and/or other components of the computer system 900).

User interface input devices 920 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 900.

User interface output devices 928 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 900 to the user or to another machine or computer system.

As further shown in FIG. 9, the storage subsystem 910 stores programming and data constructs that provide the functionality of some or all of the modules and methods described herein. These software modules are generally executed by deep learning processors 930.

Deep learning processors 930 can be graphics processing units (GPUs), field-programmable gate arrays (FPGAs), application-specific integrated circuits (ASICs), and/or coarse-grained reconfigurable architectures (CGRAs). Deep learning Processors 930 can be hosted by a deep learning cloud platform such as Google Cloud Platform™, Xilinx™, and Cirrascale™ Examples of deep learning processors 930 include Google's Tensor Processing Unit (TPU)™ rackmount solutions like GX4 Rackmount Series™, GX9 Rackmount Series™, NVIDIA DGX-1™ Microsoft′ Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

As further illustrated in FIG. 9, the memory subsystem 912 used in the storage subsystem 910 can include a number of memories including a main random access memory (RAM) 914 for storage of instructions and data during program execution and a read only memory (ROM) 916 in which fixed instructions are stored. A file storage subsystem 918 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 918 in the storage subsystem 910, or in other machines accessible by the system 100, the CPU 924, and/or the deep learning processors 930.

Additionally, the bus subsystem 922 provides a mechanism for letting the various components and subsystems of computer system 900 communicate with each other as intended. Although bus subsystem 922 is shown schematically as a single bus, alternative implementations of the bus subsystem can use multiple busses.

Computer system 900 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 900 depicted in FIG. 9 is intended only as a specific example for purposes of illustrating the preferred implementations of the present invention. Many other configurations of computer system 900 are possible having more or less components than the computer system depicted in FIG. 9.

System Overview: Poisson Generalized Linear Mixed Model of Selection

FIG. 10 is a schematic diagram of a system 1000 for detecting differential constraint via the comparison of missense-to-synonymous ratios, according to one embodiment of the disclosed technology. In some cases, the system 1000 can be the same as the system 100 introduced above in relation to FIG. 1; alternatively, the system 1000 can be a different system. The discussion of FIG. 10 is organized as follows. First, the elements of the figure are described, followed by their interconnections. Then, the use of the elements in the system 1000 is described in greater detail.

As illustrated in FIG. 10, database 1002 contains observed variant counts on a per gene basis for a target species i. As shown, the system 1000 can extract a missense-to-synonymous ratio (MSR) or, alternatively referred to as missense:synonymous ratio, for a gene g in species I from database 1002 and can fit the MSR to a Poisson generalized linear mixed model (GLMM) 1006. As further shown, database 1022 contains observed variant counts on a per gene basis for a non-target species s1, and database 1042 contains observed variant counts on a per gene basis for a non-target species s2, and so on through multiple such databases (as indicated by the ellipsis) to database 1062, which contains observed variant counts on a per gene basis for a non-target species sn. As shown, the system 1000 can aggregate non-target species {s1 . . . sn} into a cohort of non-target species, and can extract and pool variant data from databases 1002 through 1062 into a pooled MSR for gene g across non-target species s 1044. In addition, the system 1000 can fit a GLMM 1046 to the pooled MSR for gene g across non-target species s 1044 to learn a plurality of fixed effects and random effects. In some embodiments, the system 1000 can use these learned effects from GLMM 1046 to determine information related to the depletion of missense variation within the cohort of non-target species {s1 . . . sn} 1066.

As further illustrated in FIG. 10, GLMM model 1006 similarly comprises a plurality of fixed effects and random effects related to the depletion of missense variation within target species i. In addition, the system 1000 learns additional effects for the GLMM model 1006 from GLMM 1046 related to the depletion of missense variation within the cohort of non-target species {s1 . . . sn} 1066, such that GLMM 1006 approximates the relationship between the target species i MSR and the cohort of non-target species {s1 . . . sn} MSR. The system 1000 can extract a deviation metric 1008 from GLMM 1006, wherein the deviation metric 1008 corresponds to a random effect related to the deviation of the observed depletion of missense variation within target species i, as compared to what is expected based on the depletion of missense variation within the cohort of non-target species {s1 . . . sn}.

In some embodiments, the system 1000 tests the deviation metric 1008 for significance via significance test 1028. Significance test 1028 may be a Z-test, likelihood-ratio test, a t-test, or some other test, as indicated above in relation to FIG. 1. A user skilled in the art will recognize that a plurality of significance tests (with the appropriate data standardizations to meet necessary statistical assumptions) may be implemented as significance test 1028.

In applying the significance test 1028, the system 1000 can utilize a null model such that the deviation metric 1008 for target species i and the cohort of non-target species {s1 . . . sn} is equal to zero. Conversely, when applying the significance test 1028, in some embodiments, the system 1000 can utilize an alternative model such that the deviation metric 1008 for target species i and the cohort of non-target species {s1 . . . sn} is not equal to zero. As mentioned above, significance is defined by a pre-determined alpha level. If the system 1000 determines that the deviation metric 1008 for target species i and the cohort of non-target species {s1 . . . sn} is not equal to zero, the system 1000 can detect or determine differential selective constraint for gene g between target species i and non-target species {s1 . . . sn} 1048. However, if the system 1000 determines that deviation metric 1008 for target species i and the cohort of non-target species {s1 . . . sn} is not equal to zero, the system 1000 may detect or determine no differential selective constraint for gene g between target species i and non-target species {s1 . . . sn} 1049.

Further continuing with the description of system 1000, the components of the system 1000 illustrated in FIG. 10 can be implemented by software running on varying computing devices. Example computing devices for the system 10000 are a workstation, a server, a computing cluster, a blade server, a server farm, or any other data processing system or computing device. The system 1000 can also include a processing engine communicably coupled to one or more databases (e.g., the databases 1002, 1022, 1042, and/or 1062) via a network connection.

While system 1000 is described herein with reference to particular blocks, it is to be understood that the blocks are defined for convenience of description and are not intended to require a particular physical arrangement of component parts. Further, the blocks need not correspond to physically distinct components. To the extent that physical distinct components are used, connections between components can be wired and/or wireless as desired. The different elements or components can be combined into single software modules and multiple software modules can run on the same hardware.

Missense-to-Synonymous Ratio as a Measure of Selective Constraint

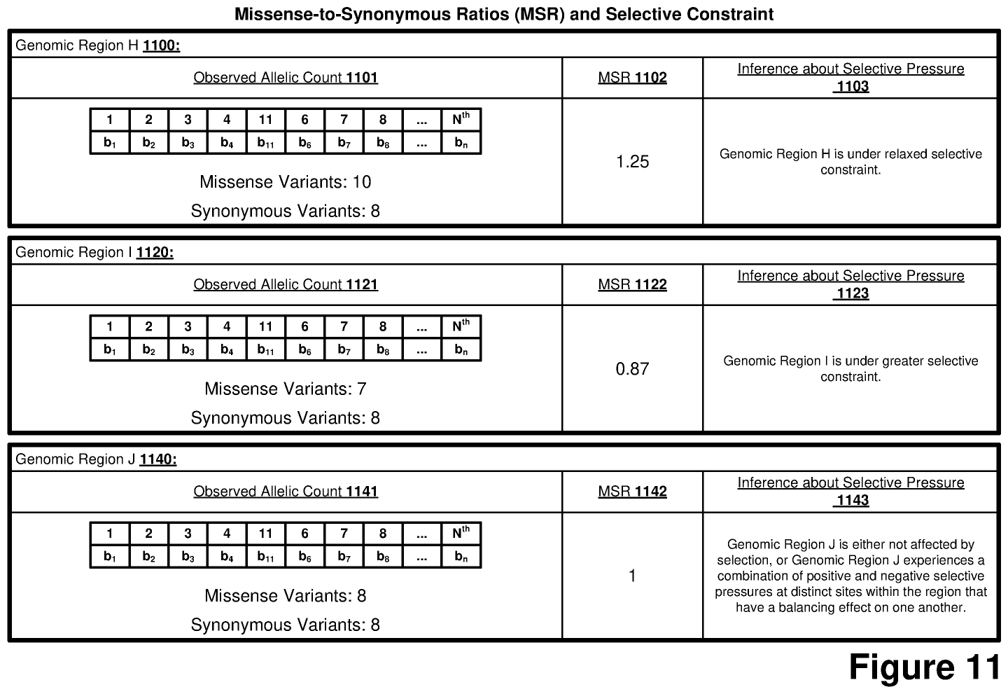

The following description of FIG. 11 through 13 refers to an embodiment of the system 1000. In some embodiments, however, the system 100 for detecting differential constraint depicted in FIG. 1 can perform the methods, determinations, operations, and functionalities described with reference to FIG. 11 through FIG. 13. FIG. 11 shows representative sample calculations of selective constraint per gene using missense-to-synonymous ratios as measures for selection. Genomic region H 1100 possesses a length of N wherein each sequence position bn comprises a nucleotide base of either thymine, adenine, guanine, or cytosine. The system 1000 can determine or identify the allelic count of segregating sites 1101 within genomic region H 1100 to include 10 missense variants and 8 synonymous variants. Thus, the system 1000 can determine that the MSR 1102 of genomic region H 1100 is equal to 1.25. Using the MSR 1102 as the measure of selection, the system 1000 can determine or infer 1103 that genomic region H 1100 is under relaxed selective constraint due to the lack of depletion of missense variants within the population.

As further illustrated in FIG. 11, genomic region 11120 possesses a length of N wherein each sequence position bn comprises a nucleotide base of either thymine, adenine, guanine, or cytosine. The system 1000 can receive data for the genomic region 11120 indicating (or analyze the genomic region 11120 to determine) an allelic count of segregating sites 1121 within genomic region 11120 to include 7 missense variants and 8 synonymous variants. Thus, the system 1000 determines that the MSR 1122 of genomic region 11120 is equal to 0.87. Using the MSR 1122 as the measure of selection, the system 1000 can determine or infer 1123 that genomic region 11120 is under greater selective constraint due to the observed depletion of missense variants within the population.

Additionally, genomic region J 1140 possesses a length of N wherein each sequence position bn comprises a nucleotide base of either thymine, adenine, guanine, or cytosine. The system 1000 can receive data indicating or determine the allelic count of segregating sites 1141 within genomic region J 1140 to include 8 missense variants and 8 synonymous variants. Thus, system 1000 can also determine that the MSR 1142 of genomic region J 1140 is equal 1. Using the MSR 1142 as the measure of selection, the system 1000 can determine or infer 1143 that genomic region J 1140 is either under neutral selection (i.e., not affected by selective pressure or constraint), or that genomic region J 1140 experiences a combination of positive and negative selective pressures at distinct sites within the region that have a balancing effect on one another.

As in above examples in FIGS. 2 through 6C, the examples given in FIG. 11 are simplified to focus on the conceptual framework underlying the relationships leveraged in the technology disclosed, wherein a user skilled in the art will recognize that while the number of variants, reported values, and required assumptions are unrealistic for real genetic populations, the concepts demonstrated are valid. The discussion now turns to the description of a GLMM to estimate depletion of missense mutation per gene per species and the use of MSR data to identify genes with differential selective constraint between species for real-world data obtained in humans and non-human primates. Because genomic populations are shaped in complex ways by sampling and demographic forces, sophisticated computations are needed.

Some implementations of the system 1000 comprise an explicit population genetic model configured to fit a curve approximating the relationship between human and primate MSR using a Poisson generalized linear mixed model and further configured to identify genes where the observed human MSR deviated significantly from what would have been expected given the gene's MSR in primates. These implementations are further configured to adjust for gene length to account for shorter genes having more variability in their MSR than longer genes.

Poisson Generalized Linear Mixed Model to Estimate Depletion of Missense Variation Per Gene

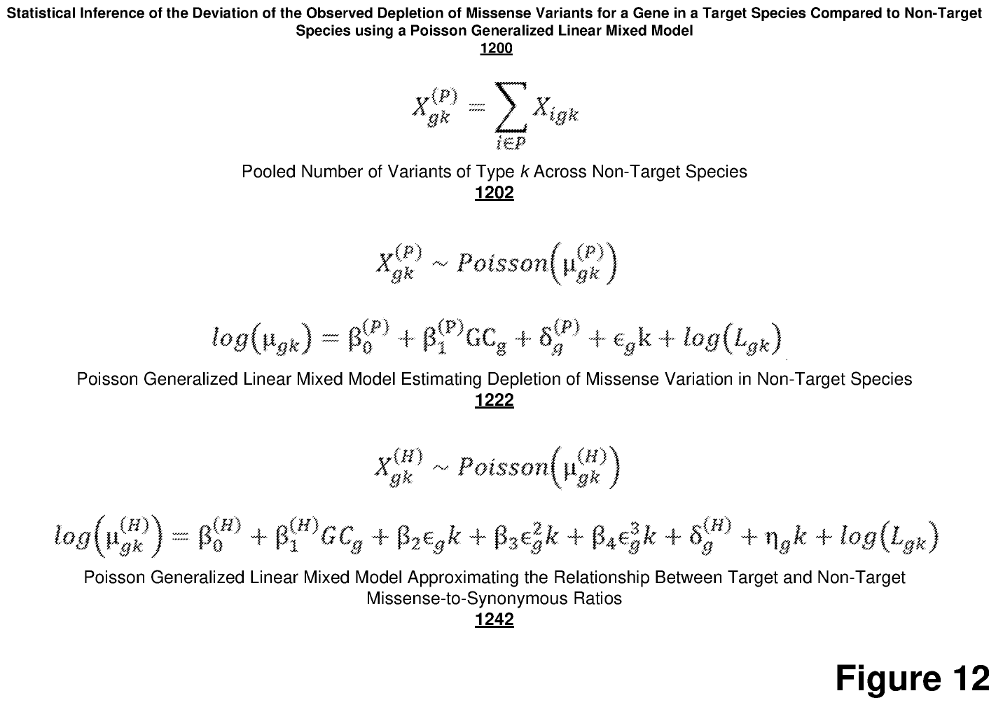

FIG. 12 shows a set of mathematical functions that the system 1000 uses to estimate the relationship between missense depletion per gene in a target species and a plurality of non-target species. To model primate variation, the system 1000 fits a model that includes a background mutation rate and the impact of GC content as fixed effects, and that includes random effects accounting for gene-specific mutation rates and the depletion of nonsynonymous variation. Recalling from the population genetic model as described in relation to FIG. 7A that X_{igk} is the number of mutations of type k (k=0 is synonymous, k=1 is missense) in gene g of species i, the system 1000 can aggregate all non-human primates into a single cohort to get the total number of variants of type k in gene g across non-human primates, 1202. As shown, P is the set of non-human primates, and the superscript (P) indicates that the value is over non-human primates and is not an exponent.

As further illustrated in FIG. 12, the system 1000 can model X_{gk}^{(P)} as a Poisson GLMM 1222 where β₀^{(P)} is a fixed effect corresponding to background mutation rate, where β₁^{(P)} is a fixed effect that corresponds to the impact of GC content, δ_{g}^{(P)} is a random effect corresponding to the discrepancy between the mutation rate of gene g and the genome-wide background, ∈_{g} is a random effect corresponding to the deficit of missense variation, and where L_{gk} is the number of sites of type k in gene g.

As shown, the system 1000 can determine and apply the estimates of ∈_{g} to build model 1242 for the human data X_{gk}^{(H)} ≡X_{hgk}. In some embodiments, the terms or parameters of the model 1242, β₁^{(H)}, β₂^{(H)}, δ_{g}^{(H)}, and L_{gk} have the same interpretation as described above for primate model 1222. However, the system 1000 applies the parameters of the model 1242 to human data. As shown, the system 1000 determines, detects, or identifies additional fixed effects β₂, β₃, and β₄ in 1242, where the additional fixed effects model a nonlinear relationship between the missense depletion in primates and the missense depletion in humans. Thus, the system 1000 can utilize the remaining random effect, η_{g}, as the deviation of the observed depletion of missense variants in humans compared to what would be expected based on the primate depletion. In particular, the system 1000 determines that η_{g}<0 indicates that a gene has even fewer missense variants than would be expected based on primates. Consequently, the system 1000 thus determines that there is stronger constraint in humans than in primates. Conversely, the system 1000 can determine that η_{g}>0 indicates an excess of missense variants compared to the expectation based on primates, and the system 1000 thus determines that there is relaxed constraint in humans compared to primates.

Identifying Differential Selective Constraint Between Target and Non-Target Species Leveraging Depletion of Missense Variation

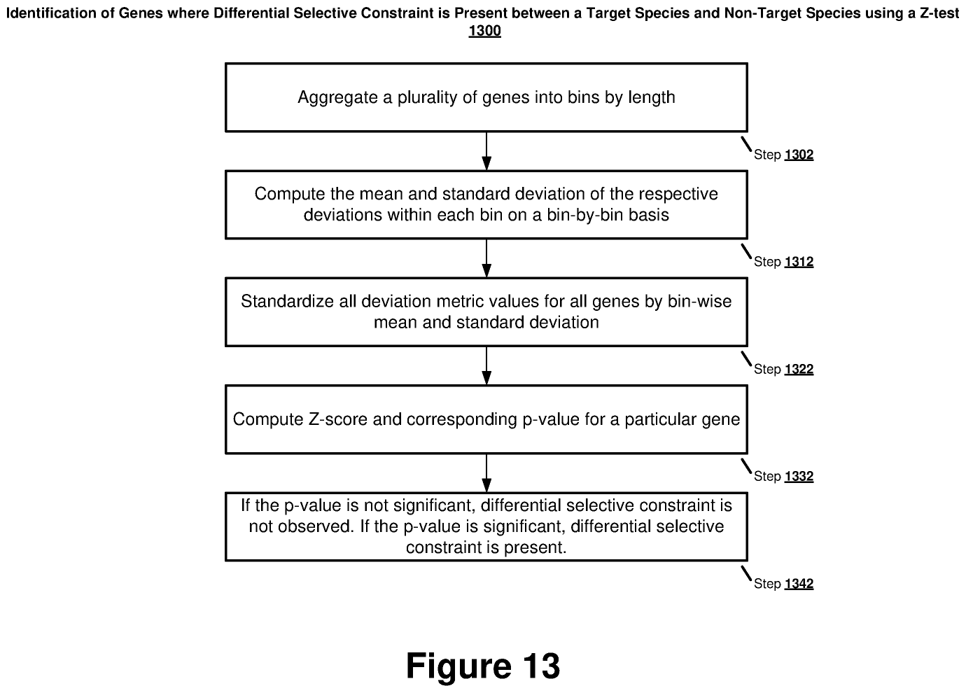

FIG. 13 shows a flow diagram comprising steps that the system 1000 performs to identify genes where differential selective constraint is present between a target species and one or more non-target species using a Z-test. To determine which η_{g} values were significantly different from 0, and hence indicative of human having less or more constraint than non-human primates, the system 1000 controls for gene length by binning genes by gene length and creating Z-scores based on the η_{g} within each bin. In some embodiments, shorter genes are likely to have a large magnitude of η_{g} by chance. Moreover, because it is not expected that selection is identical between humans and primates, the system 1000 can use the Z-scores to identify genes for which η_{g} significantly deviates from a background distribution.

As illustrated in FIG. 13, the system 1000 performs a number of steps or operations. First, the system 1000 performs step 1302 to aggregate a plurality of genes into a number of bins (e.g., by quantiles) by the number of amino acids in the human reference genome. In addition, the system 1000 performs step 1312 to determine or compute the mean and standard deviation of Ig values within each bin on a bin-by-bin basis. Further, the system 1000 performs step 1322 to standardize all values using the computed mean and standard deviation per bin. As shown, the system 1000 also performs step 1332 to determine or compute a Z-score can, along with the corresponding p-values, where the Z-score follows the standard normal distribution Z˜N (0,1). Lastly, as shown in FIG. 13, the system 1000 performs step 1342 to determine if a particular η_{g} value for a particular gene indicates differential constraint between humans and non-human primates, per the significance of the p-value at a set alpha level.

Computer System for Implementation of a Poisson Generalized Linear Mixed Model of Selection

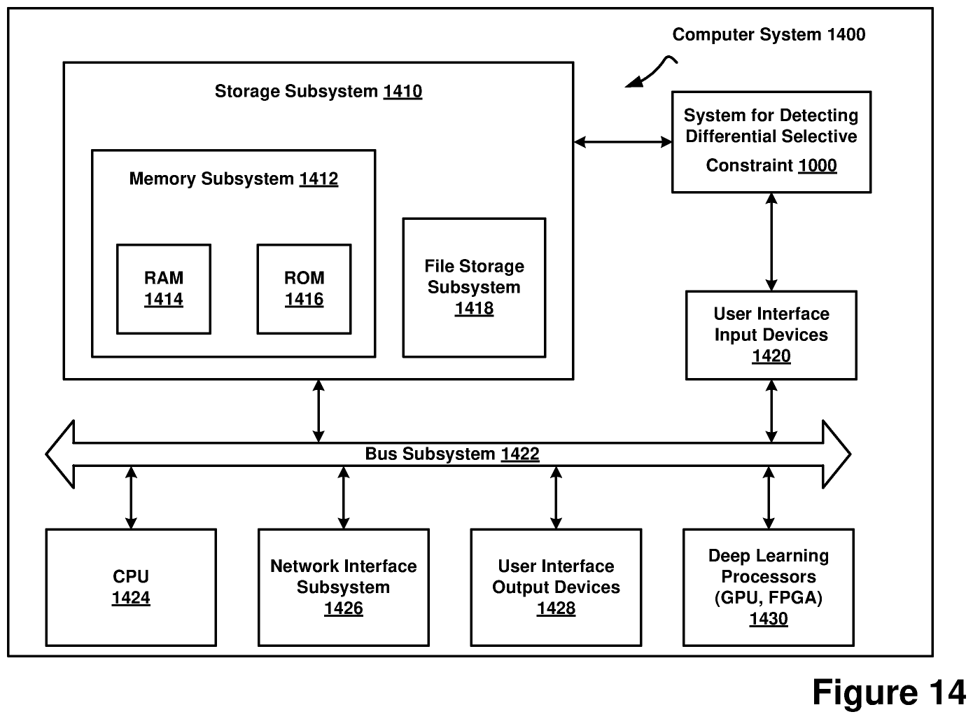

FIG. 14 shows a second example computer system (e.g., for the system 1000) that can be used to implement the technology disclosed, according to one embodiment of the disclosed technology. Computer system 1400 can facilitate or perform the actions or operations described herein relating to the system 1000 (e.g., the system for detecting differential selective constraint). Indeed, as shown, the computer system 900 can house or store the system 1000. As further shown, the computer system 900 includes at least one central processing unit (CPU) 1424 that communicates with a number of peripheral devices via bus subsystem 1422. These peripheral devices can include a storage subsystem 1410 including, for example, memory devices and a file storage subsystem 1418, user interface input devices 1420, user interface output devices 1428, and a network interface subsystem 1426. The input and output devices allow user interaction with computer system 1400. Network interface subsystem 1426 provides an interface to outside networks, including an interface to corresponding interface devices in other computer systems.

In one implementation, the system 1000 is communicably linked to the storage subsystem 1410 and the user interface input devices 1420 (and/or other components of the computer system 1400).

User interface input devices 1420 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 1400.

User interface output devices 1428 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 1400 to the user or to another machine or computer system.

As further shown in FIG. 14, storage subsystem 1410 stores programming and data constructs that provide the functionality of some or all of the modules and methods described herein. These software modules are generally executed by deep learning processors 1430.

Deep learning processors 1430 can be graphics processing units (GPUs), field-programmable gate arrays (FPGAs), application-specific integrated circuits (ASICs), and/or coarse-grained reconfigurable architectures (CGRAs). Deep learning Processors 1430 can be hosted by a deep learning cloud platform such as Google Cloud Platform™, Xilinx™, and Cirrascale™ Examples of deep learning processors 1430 include Google's Tensor Processing Unit (TPU)™ rackmount solutions like GX4 Rackmount Series™, GX14 Rackmount Series™, NVIDIA DGX-1™ Microsoft′ Stratix V FPGA™, Graphcore's Intelligent Processor Unit (IPU)™, Qualcomm's Zeroth Platform™ with Snapdragon Processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, TBM TrueNorth™, Lambda GPU Server with Testa V100s™, and others.

As further illustrated in FIG. 14, the memory subsystem 1412 used in the storage subsystem 1410 can include a number of memories including a main random access memory (RAM) 1414 for storage of instructions and data during program execution and a read only memory (ROM) 1416 in which fixed instructions are stored. A file storage subsystem 1418 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 1418 in the storage subsystem 1410, or in other machines accessible by the system 1000, the CPU 924, and/or the deep learning processors 930.

Additionally, the bus subsystem 1422 provides a mechanism for letting the various components and subsystems of computer system 1400 communicate with each other as intended. Although bus subsystem 1422 is shown schematically as a single bus, alternative implementations of the bus subsystem can use multiple busses.

Computer system 1400 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 1400 depicted in FIG. 14 is intended only as a specific example for purposes of illustrating the preferred implementations of the present invention. Many other configurations of computer system 1400 are possible having more or less components than the computer system depicted in FIG. 14.

Performance Measure Results as Objective Indicia of Non-Obviousness and Inventiveness

The discussion thus far has covered a plurality of implementations of the technology disclosed for identifying genes with differential selective constraint between a target species and a plurality of non-target species. The discussion now turns to performance results of various implementations of the technology disclosed.

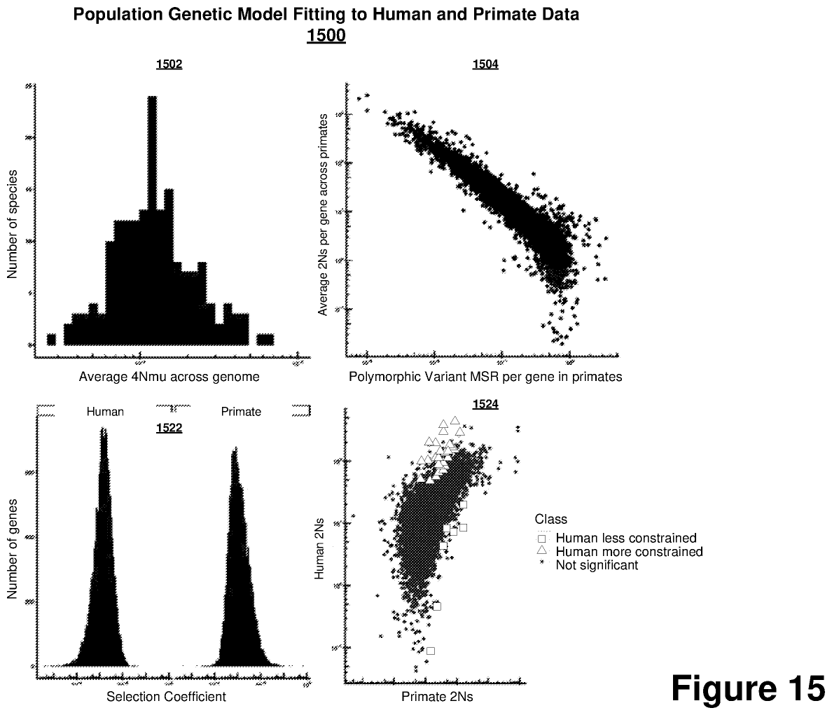

FIG. 15 shows a plurality of performance metrics of the system 100 and/or the system 1000 in applying a population genetic model fitting to human and primate data 1500. Histogram 1502 shows the distribution of population-scaled mutation rates across a plurality of primate species. Graph 1504 shows the correlation between a pooled primate MSR and inferred selection. The x-axis shows the MSR for each gene when pooled across all non-human primates. Indeed, as suggested above, the system 100 can pool or share information across multiple genes when determining missense-to-synonymous ratios, and the graph 1504 illustrates relationships between missense-to-synonymous ratios for each gene pooled across non-human primates (e.g., non-target species) and inferred selection (e.g., determining whether selection occurred on the gene). The y-axis shows the inferred 2Ns for each gene. Histogram 1522 shows the distribution of fitness effects across genes in humans and non-human primates. Histogram 1522 is over all genes that pass the filters to be used in the analysis. Graph 1524 shows the correlation between human selection and primate selection. The x-axis shows the strength of selection in humans, and y-axis shows the strength of selection in primates. Highlighted points (e.g., points indicated by squares and/or triangles in within the graph 1524) are significant according to point the population genetic model and the MSR regression.

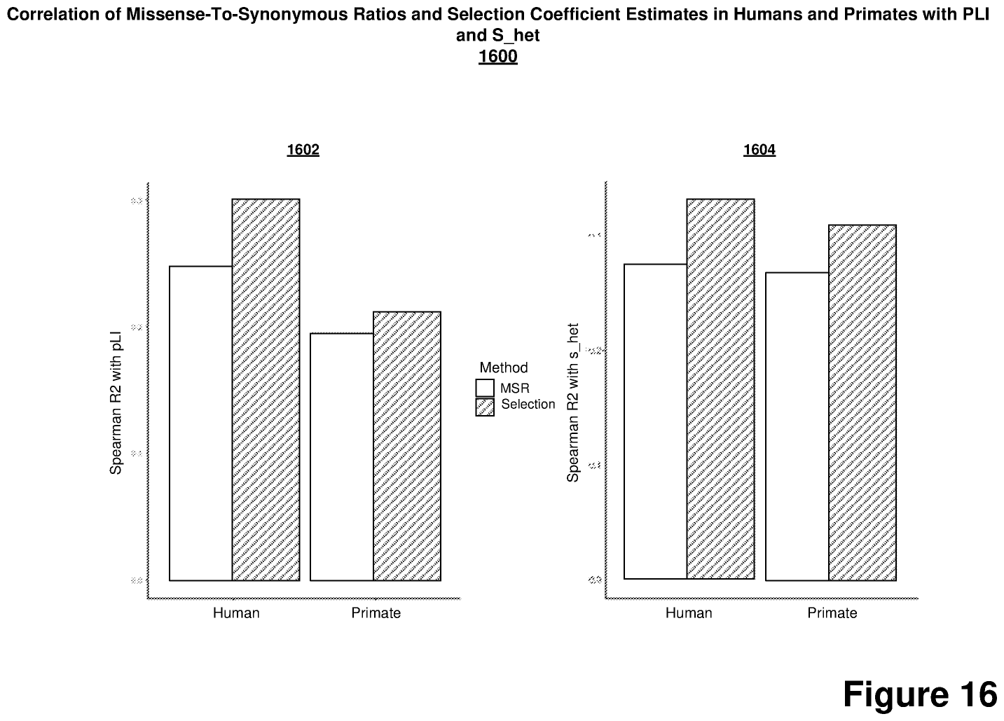

FIG. 16 shows the correlation 1600 of missense-to-synonymous ratios and selection coefficient estimates in humans and primates with pLI and s_het. Specifically, bar chart 1602 shows the correlation of missense-to-synonymous ratios and selection coefficient estimates in humans and primates with pLI, wherein the left (white) bars represent the raw MSR of polymorphic variants and the right (patterned) bars show estimated selection coefficients. Bars are grouped by whether they are based on human data or pooled primate data. Bar chart 1604 shows the correlation of missense-to-synonymous ratios and selection coefficient estimates in humans and primates with s_het. As in 1602, the left (white) bars represent the raw MSR of polymorphic variants and the right (patterned) bars in the bar chart 1604 show estimated selection coefficients. Bars are grouped by whether they are based on human data or pooled primate data.

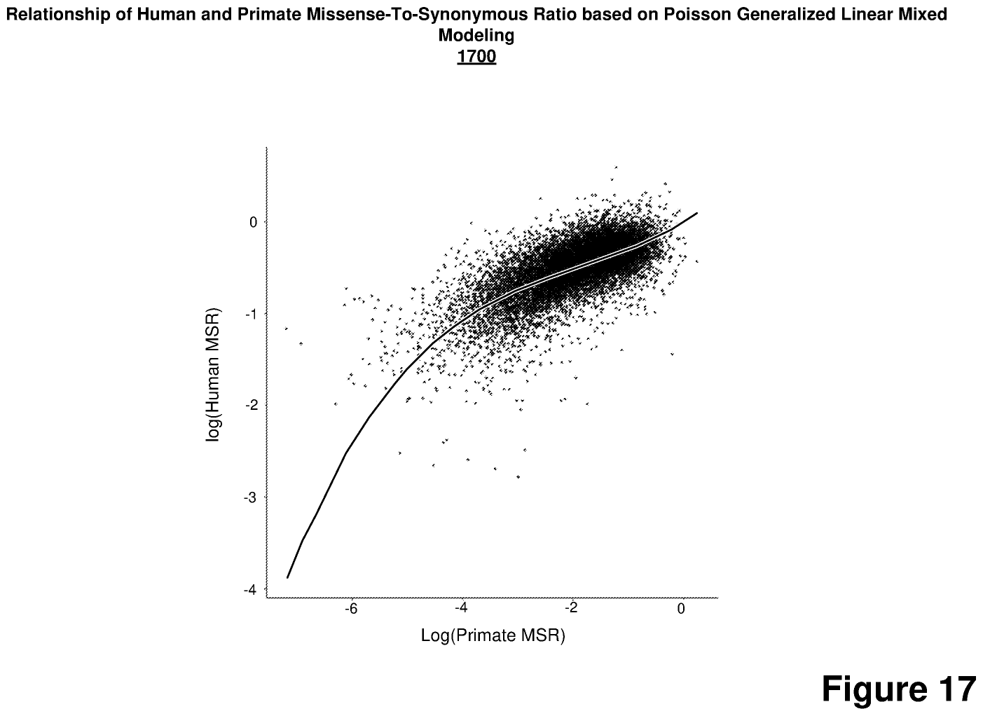

FIG. 17 shows the relationship 1700 of human and primate missense-to-synonymous ratio based on Poisson generalized linear mixed modeling. The x-axis shows the log-scaled MSR among polymorphic variants in the cohort of primates compared. The y-axis shows the log-scaled MSR among polymorphic variants in humans. The line of best fit through the scatterplot represents the best-fit relationship as inferred by the model.

### CLAUSES

The technology disclosed, in particularly, the clauses disclosed in this section, can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

1. A computer-implemented method of detecting differential selective constraint for a gene of interest between a target species and one or more non-target species, the method comprising:
determining a target species background distribution of mutation rates in the gene of interest;
applying the target species background distribution of mutation rates in the gene of interest to estimate a target species selection coefficient for the gene of interest;
for at least one non-target species:
   determining a non-target species background distribution of mutation rates in the gene of interest, wherein the non-target species background distribution of mutation rates in the gene of interest is determined per-species,
   applying the non-target species background distribution of mutation rates in the gene of interest to estimate a non-target species selection coefficient for the gene of interest, wherein the non-target species selection coefficient for the gene of interest is determined per-species, and computing an average non-target species selection coefficient for the gene of interest; and
comparing the target species selection coefficient for the gene of interest and the average non-target species selection coefficient for the gene of interest to detect if differential selective constraint is present for the gene of interest between the target species and the one or more non-target species.
   2. The computer-implemented method of clause 1, wherein a particular background distribution of mutation rates in the gene of interest for a particular species is determined by fitting a Poisson Random Field model for a Poisson random variable.
   3. The computer-implemented method of clause 2, wherein an observed count of segregating sites in the gene of interest in the particular species is the Poisson random variable with a mean value determined by a mutation rate, a demography, and a sample size.
   4. The computer-implemented method of clause 1, further comprising determining a distribution of mutation rates for segregating synonymous sites in the gene of interest per-species, wherein the distribution of mutation rates for segregating synonymous sites in the gene of interest per-species is substituted for an inferred distribution of mutation rates for segregating nonsynonymous sites in the gene of interest per-species.
   5. The computer-implemented method of clause 1, wherein the target species is homologous to the one or more non-target species and the gene of interest is homologous between the target species and the one or more non-target species.
   6. The computer-implemented method of clause 1, wherein the target species is a human and at least one non-target species is a non-human primate.
   7. The computer-implemented method of clause 1, wherein the average non-target species selection coefficient for the gene of interest for a singular non-target species is equal to the non-target species selection coefficient for the gene of interest for the singular non-target species, and wherein the average non-target species selection coefficient for the gene of interest for a plurality of non-target species is equal to a mean value across a plurality of non-target species selection coefficients per-species for the plurality of non-target species.
   8. The computer-implemented method of clause 7, further comprising a correction of the average non-target species selection coefficient for the gene of interest for demographic differences.
   9. The computer-implemented method of clause 1, wherein comparison of the target species selection coefficient for the gene of interest and the average non-target species selection coefficient for the gene of interest comprises a test for statistical significance.
   10. A computer-implemented method for identifying genes with differential selective constraint between a target species and a plurality of non-target species, the method comprising:
obtaining a plurality of missense-to-synonymous ratios at per-gene, per-species resolution,
   wherein a particular missense-to-synonymous ratio within the plurality of missense-to-synonymous ratios is a proxy for selective constraint on a particular gene in a particular species;
pooling a plurality of missense-to-synonymous ratios at per-gene, per-species resolution for a plurality of non-target species to obtain a pooled non-target species missense-to-synonymous ratio at per-gene resolution; and
for at least one gene of interest:
   processing the pooled non-target species missense-to-synonymous ratio for the gene of interest to estimate a non-target species depletion of missense variation in the gene of interest,
   processing a target species missense-to-synonymous ratio for the gene of interest to estimate a target species depletion of missense variation in the gene of interest,
   computing a deviation metric to quantify a magnitude and a direction of deviation of the target species depletion of missense variation in the gene of interest from the non-target species depletion of missense variation in the gene of interest, and
   leveraging the deviation metric to approximate a magnitude and a direction of differential selective constraint between the target species and the plurality of non-target species.
      11. The computer-implemented method of clause 10, further including fitting a Poisson generalized linear mixed model, comprising one or more fixed effects, to the particular missense-to-synonymous ratio for the particular gene in the particular species to estimate the depletion of missense variation in the particular gene in the particular species.
      12. The computer-implemented method of clause 11, further including:
fitting a first Poisson generalized linear mixed model to estimate a first depletion of missense variation in the gene of interest in a first group comprising at least one species,
fitting a second Poisson generalized linear mixed model to estimate a second depletion of missense variation in the gene of interest in a second group comprising at least one species,
incorporating the first depletion of missense variation in the gene of interest from the first Poisson generalized linear mixed model into the second Poisson generalized mixed model to generate a nonlinear relationship model between the first depletion of missense variation in the gene of interest and the second depletion of missense variation in the gene of interest, and
estimating the deviation metric for the first group and the second group from the nonlinear relationship model between the first depletion of missense variation in the gene of interest and the second depletion of missense variation in the gene of interest.
   13. The computer-implemented method of clause 10, wherein a magnitude of the missense-to-synonymous ratio for the gene of interest in a particular species is inversely proportional to a magnitude of selective constraint on the gene of interest in the particular species.
   14. The computer-implemented method of clause 10, wherein the target species is homologous to the plurality of non-target species and the gene of interest is homologous between the target species and the plurality of non-target species.
   15. The computer-implemented method of clause 14, wherein the target species is a human and the plurality of non-target species are non-human primates.
   16. The computer-implemented method of clause 10, wherein a negative value for the deviation metric for the gene of interest has a negative direction and a positive value for the deviation metric for the gene of interest has a positive direction, and wherein a negative direction for the deviation metric for the gene of interest indicates a stronger selective constraint in the target species relative to the selective constraint in the plurality of non-target species and a positive direction for the deviation metric for the gene of interest indicates a weaker selective constraint in the target species relative to the selective constraint in the plurality of non-target species.
   17. The computer-implemented method of clause 16, wherein a significance statistic for the deviation metric for the gene of interest identifies differential constraint on the gene of interest, and wherein computing the significance statistic for the deviation metric for the gene of interest comprises:
aggregating a plurality of genes by length,
   wherein one gene within the plurality of genes is the gene of interest,
computing a mean and a standard deviation of a plurality of deviation metrics for the plurality of genes on a bin-by-bin basis, and
performing a significance test for the deviation metric for the gene of interest using the mean and the standard deviation of the bin containing the gene of interest.
   18. A system including one or more processors coupled to memory, the memory loaded with computer instructions to detect differential selective constraint for a gene of interest between a target species and one or more non-target species, the instructions, when executed on the processors, implement actions comprising:
determining a target species background distribution of mutation rates in the gene of interest;
applying the target species background distribution of mutation rates in the gene of interest to estimate a target species selection coefficient for the gene of interest;
for at least one non-target species:
   determining a non-target species background distribution of mutation rates in the gene of interest, wherein the non-target species background distribution of mutation rates in the gene of interest is determined per-species,
   applying the non-target species background distribution of mutation rates in the gene of interest to estimate a non-target species selection coefficient for the gene of interest, wherein the non-target species selection coefficient for the gene of interest is determined per-species, and
computing an average non-target species selection coefficient for the gene of interest; and comparing the target species selection coefficient for the gene of interest and the average non-target species selection coefficient for the gene of interest to detect if differential selective constraint is present for the gene of interest between the target species and the one or more non-target species.
   19. A non-transitory computer readable storage medium impressed with computer program instructions to detect differential selective constraint for a gene of interest between a target species and one or more non-target species, the instructions, when executed on a processor, implement a method comprising:
determining a target species background distribution of mutation rates in the gene of interest;
applying the target species background distribution of mutation rates in the gene of interest to estimate a target species selection coefficient for the gene of interest;
for at least one non-target species:
   determining a non-target species background distribution of mutation rates in the gene of interest, wherein the non-target species background distribution of mutation rates in the gene of interest is determined per-species,
   applying the non-target species background distribution of mutation rates in the gene of interest to estimate a non-target species selection coefficient for the gene of interest, wherein the non-target species selection coefficient for the gene of interest is determined per-species, and computing an average non-target species selection coefficient for the gene of interest; and
comparing the target species selection coefficient for the gene of interest and the average non-target species selection coefficient for the gene of interest to detect if differential selective constraint is present for the gene of interest between the target species and the one or more non-target species.
   20. A system including one or more processors coupled to memory, the memory loaded with computer instructions to identify genes with differential selective constraint between a target species and a plurality of non-target species, the instructions, when executed on the processors, implement actions comprising:
obtaining a plurality of missense-to-synonymous ratios at per-gene, per-species resolution,
   wherein a particular missense-to-synonymous ratio within the plurality of missense-to-synonymous ratios is a proxy for selective constraint on a particular gene in a particular species;
pooling a plurality of missense-to-synonymous ratios at per-gene, per-species resolution for a plurality of non-target species to obtain a pooled non-target species missense-to-synonymous ratio at per-gene resolution; and
for at least one gene of interest:
   processing the pooled non-target species missense-to-synonymous ratio for the gene of interest to estimate a non-target species depletion of missense variation in the gene of interest,
   processing a target species missense-to-synonymous ratio for the gene of interest to estimate a target species depletion of missense variation in the gene of interest,
   computing a deviation metric to quantify a magnitude and a direction of deviation of the target species depletion of missense variation in the gene of interest from the non-target species depletion of missense variation in the gene of interest, and
      leveraging the deviation metric to approximate a magnitude and a direction of differential selective constraint between the target species and the plurality of non-target species.
      21. A non-transitory computer readable storage medium impressed with computer program instructions to identify genes with differential selective constraint between a target species and a plurality of non-target species, the instructions, when executed on a processor, implement a method comprising:
obtaining a plurality of missense-to-synonymous ratios at per-gene, per-species resolution,
   wherein a particular missense-to-synonymous ratio within the plurality of missense-to-synonymous ratios is a proxy for selective constraint on a particular gene in a particular species;
pooling a plurality of missense-to-synonymous ratios at per-gene, per-species resolution for a plurality of non-target species to obtain a pooled non-target species missense-to-synonymous ratio at per-gene resolution; and
for at least one gene of interest:
   processing the pooled non-target species missense-to-synonymous ratio for the gene of interest to estimate a non-target species depletion of missense variation in the gene of interest,
   processing a target species missense-to-synonymous ratio for the gene of interest to estimate a target species depletion of missense variation in the gene of interest,
   computing a deviation metric to quantify a magnitude and a direction of deviation of the target species depletion of missense variation in the gene of interest from the non-target species depletion of missense variation in the gene of interest, and
      leveraging the deviation metric to approximate a magnitude and a direction of differential selective constraint between the target species and the plurality of non-target species.

What we claim is:

1. A computer-implemented method of detecting differential selective constraint for a gene of interest between a target species and one or more non-target species, the computer-implemented method comprising:
determining a target species background distribution of mutation rates in the gene of interest;
applying the target species background distribution of mutation rates in the gene of interest to estimate a target species selection coefficient for the gene of interest;
for at least one non-target species:
determining a non-target species background distribution of mutation rates in the gene of interest, wherein the non-target species background distribution of mutation rates in the gene of interest is determined per-species,
applying the non-target species background distribution of mutation rates in the gene of interest to estimate a non-target species selection coefficient for the gene of interest, wherein the non-target species selection coefficient for the gene of interest is determined per-species, and
computing an average non-target species selection coefficient for the gene of interest; and
comparing the target species selection coefficient for the gene of interest and the average non-target species selection coefficient for the gene of interest to detect if differential selective constraint is present for the gene of interest between the target species and the one or more non-target species.

2. The computer-implemented method of claim 1, wherein a particular background distribution of mutation rates in the gene of interest for a particular species is determined by fitting a Poisson Random Field model for a Poisson random variable.

3. The computer-implemented method of claim 2, wherein an observed count of segregating sites in the gene of interest in the particular species is the Poisson random variable with a mean value determined by a mutation rate, a demography, and a sample size.

4. The computer-implemented method of claim 1, further comprising determining a distribution of mutation rates for segregating synonymous sites in the gene of interest per-species, wherein the distribution of mutation rates for segregating synonymous sites in the gene of interest per-species is substituted for an inferred distribution of mutation rates for segregating nonsynonymous sites in the gene of interest per-species.

5. The computer-implemented method of claim 1, wherein the target species is homologous to the one or more non-target species and the gene of interest is homologous between the target species and the one or more non-target species.

6. The computer-implemented method of claim 1, wherein the target species is a human and at least one non-target species is a non-human primate.

7. The computer-implemented method of claim 1, wherein the average non-target species selection coefficient for the gene of interest for a singular non-target species is equal to the non-target species selection coefficient for the gene of interest for the singular non-target species, and wherein the average non-target species selection coefficient for the gene of interest for a plurality of non-target species is equal to a mean value across a plurality of non-target species selection coefficients per-species for the plurality of non-target species.

8. The computer-implemented method of claim 7, further comprising a correction of the average non-target species selection coefficient for the gene of interest for demographic differences.

9. The computer-implemented method of claim 1, wherein comparison of the target species selection coefficient for the gene of interest and the average non-target species selection coefficient for the gene of interest comprises a test for statistical significance.

10. A computer-implemented method for identifying genes with differential selective constraint between a target species and a plurality of non-target species, the computer-implemented method comprising:
obtaining a plurality of missense-to-synonymous ratios at per-gene, per-species resolution,
wherein a particular missense-to-synonymous ratio within the plurality of missense-to-synonymous ratios is a proxy for selective constraint on a particular gene in a particular species;
pooling a plurality of missense-to-synonymous ratios at per-gene, per-species resolution for a plurality of non-target species to obtain a pooled non-target species missense-to-synonymous ratio at per-gene resolution; and
for at least one gene of interest:
processing the pooled non-target species missense-to-synonymous ratio for the gene of interest to estimate a non-target species depletion of missense variation in the gene of interest,
processing a target species missense-to-synonymous ratio for the gene of interest to estimate a target species depletion of missense variation in the gene of interest,
computing a deviation metric to quantify a magnitude and a direction of deviation of the target species depletion of missense variation in the gene of interest from the non-target species depletion of missense variation in the gene of interest, and
leveraging the deviation metric to approximate a magnitude and a direction of differential selective constraint between the target species and the plurality of non-target species.

11. The computer-implemented method of claim 10, further including fitting a Poisson generalized linear mixed model, comprising one or more fixed effects, to the particular missense-to-synonymous ratio for the particular gene in the particular species to estimate the depletion of missense variation in the particular gene in the particular species.

12. The computer-implemented method of claim 11, further including:
fitting a first Poisson generalized linear mixed model to estimate a first depletion of missense variation in the gene of interest in a first group comprising at least one species,
fitting a second Poisson generalized linear mixed model to estimate a second depletion of missense variation in the gene of interest in a second group comprising at least one species,
incorporating the first depletion of missense variation in the gene of interest from the first Poisson generalized linear mixed model into the second Poisson generalized linear mixed model to generate a nonlinear relationship model between the first depletion of missense variation in the gene of interest and the second depletion of missense variation in the gene of interest, and
estimating the deviation metric for the first group and the second group from the nonlinear relationship model between the first depletion of missense variation in the gene of interest and the second depletion of missense variation in the gene of interest.

13. The computer-implemented method of claim 10, wherein a magnitude of the missense-to-synonymous ratio for the gene of interest in a particular species is inversely proportional to a magnitude of selective constraint on the gene of interest in the particular species.

14. The computer-implemented method of claim 10, wherein the target species is homologous to the plurality of non-target species and the gene of interest is homologous between the target species and the plurality of non-target species.

15. The computer-implemented method of claim 14, wherein the target species is a human and the plurality of non-target species are non-human primates.

16. The computer-implemented method of claim 10, wherein a negative value for the deviation metric for the gene of interest has a negative direction and a positive value for the deviation metric for the gene of interest has a positive direction, and wherein a negative direction for the deviation metric for the gene of interest indicates a stronger selective constraint in the target species relative to the selective constraint in the plurality of non-target species and a positive direction for the deviation metric for the gene of interest indicates a weaker selective constraint in the target species relative to the selective constraint in the plurality of non-target species.

17. The computer-implemented method of claim 16, wherein a significance statistic for the deviation metric for the gene of interest identifies differential constraint on the gene of interest, and wherein computing the significance statistic for the deviation metric for the gene of interest comprises:
aggregating a plurality of genes by length,
wherein one gene within the plurality of genes is the gene of interest,
computing a mean and a standard deviation of a plurality of deviation metrics for the plurality of genes on a bin-by-bin basis, and
performing a significance test for the deviation metric for the gene of interest using the mean and the standard deviation of the bin containing the gene of interest.

18. A system including one or more processors coupled to memory, the memory loaded with computer instructions to detect differential selective constraint for a gene of interest between a target species and one or more non-target species, the computer instructions, when executed on the one or more processors, implement actions comprising:
determining a target species background distribution of mutation rates in the gene of interest;
applying the target species background distribution of mutation rates in the gene of interest to estimate a target species selection coefficient for the gene of interest;
for at least one non-target species:
determining a non-target species background distribution of mutation rates in the gene of interest, wherein the non-target species background distribution of mutation rates in the gene of interest is determined per-species,
applying the non-target species background distribution of mutation rates in the gene of interest to estimate a non-target species selection coefficient for the gene of interest, wherein the non-target species selection coefficient for the gene of interest is determined per-species, and
computing an average non-target species selection coefficient for the gene of interest; and
comparing the target species selection coefficient for the gene of interest and the average non-target species selection coefficient for the gene of interest to detect if differential selective constraint is present for the gene of interest between the target species and the one or more non-target species.

19. A non-transitory computer readable storage medium impressed with computer program instructions to detect differential selective constraint for a gene of interest between a target species and one or more non-target species, the computer program instructions, when executed on a processor, implement a method comprising:
determining a target species background distribution of mutation rates in the gene of interest;
applying the target species background distribution of mutation rates in the gene of interest to estimate a target species selection coefficient for the gene of interest;
for at least one non-target species:
determining a non-target species background distribution of mutation rates in the gene of interest, wherein the non-target species background distribution of mutation rates in the gene of interest is determined per-species,
applying the non-target species background distribution of mutation rates in the gene of interest to estimate a non-target species selection coefficient for the gene of interest, wherein the non-target species selection coefficient for the gene of interest is determined per-species, and
computing an average non-target species selection coefficient for the gene of interest; and
comparing the target species selection coefficient for the gene of interest and the average non-target species selection coefficient for the gene of interest to detect if differential selective constraint is present for the gene of interest between the target species and the one or more non-target species.

20. A system including one or more processors coupled to memory, the memory loaded with computer instructions to identify genes with differential selective constraint between a target species and a plurality of non-target species, the computer instructions, when executed on the one or more processors, implement actions comprising:
obtaining a plurality of missense-to-synonymous ratios at per-gene, per-species resolution,
wherein a particular missense-to-synonymous ratio within the plurality of missense-to-synonymous ratios is a proxy for selective constraint on a particular gene in a particular species;
pooling a plurality of missense-to-synonymous ratios at per-gene, per-species resolution for a plurality of non-target species to obtain a pooled non-target species missense-to-synonymous ratio at per-gene resolution; and
for at least one gene of interest:
processing the pooled non-target species missense-to-synonymous ratio for the gene of interest to estimate a non-target species depletion of missense variation in the gene of interest,
processing a target species missense-to-synonymous ratio for the gene of interest to estimate a target species depletion of missense variation in the gene of interest,
computing a deviation metric to quantify a magnitude and a direction of deviation of the target species depletion of missense variation in the gene of interest from the non-target species depletion of missense variation in the gene of interest, and
leveraging the deviation metric to approximate a magnitude and a direction of differential selective constraint between the target species and the plurality of non-target species.

21. A non-transitory computer readable storage medium impressed with computer program instructions to identify genes with differential selective constraint between a target species and a plurality of non-target species, the computer program instructions, when executed on a processor, implement a method comprising:
obtaining a plurality of missense-to-synonymous ratios at per-gene, per-species resolution,
wherein a particular missense-to-synonymous ratio within the plurality of missense-to-synonymous ratios is a proxy for selective constraint on a particular gene in a particular species;
pooling a plurality of missense-to-synonymous ratios at per-gene, per-species resolution for a plurality of non-target species to obtain a pooled non-target species missense-to-synonymous ratio at per-gene resolution; and
for at least one gene of interest:
processing the pooled non-target species missense-to-synonymous ratio for the gene of interest to estimate a non-target species depletion of missense variation in the gene of interest,
processing a target species missense-to-synonymous ratio for the gene of interest to estimate a target species depletion of missense variation in the gene of interest,
computing a deviation metric to quantify a magnitude and a direction of deviation of the target species depletion of missense variation in the gene of interest from the non-target species depletion of missense variation in the gene of interest, and
leveraging the deviation metric to approximate a magnitude and a direction of differential selective constraint between the target species and the plurality of non-target species.
